(19) United States
(12) Patent Application Publication
(10) Pub. No.: US 2022/0125886 A1
(43) Pub. Date: Apr. 28, 2022

(54) CO-FORMULATIONS OF AMYLIN ANALOGUES WITH INSULIN ANALOGUES FOR TREATMENT OF DIABETES

(71) Applicant: The Board of Trustees of the Leland Stanford Junior University, Palo Alto, CA (US)

(72) Inventors: Eric A. APPEL, Palo Alto, CA (US); Bruce A. BUCKINGHAM, Palo Alto, CA (US); David M. MAAHS, Aurora, CO (US); Caitlin MAIKAWA, Palo Alto, CA (US); Gillie Agmon ROTH, Stanford, CA (US)

(73) Assignee: The Board of Trustees of the Leland Stanford Junior University, Palo Alto, CA (US)

(21) Appl. No.: 17/430,664

(22) PCT Filed: Feb. 12, 2020
(86) PCT No.: PCT/US2020/017997

§ 371 Date: Aug. 12, 2021

Related U.S. Application Data
(60) Provisional application No. 62/804,357, filed on Feb. 12, 2019.

(51) Int. Cl. A61K 38/28; A61P 3/10; A61K 47/60

(52) U.S. Cl. CPC A61K 38/28; A61K 47/60; A61P 3/10

(57) ABSTRACT

Compositions and methods for treating diabetes are disclosed. In particular, the invention relates to co-formulations of amylin analogues with insulin analogues for treatment of diabetes.

### RELATED APPLICATIONS

This application claims the benefit of the priority of U.S. Provisional Application No. 62/804,357, filed Feb. 12, 2019, which is incorporated herein by reference in its entirety.

### TECHNICAL FIELD

The present invention pertains to co-formulations of amylin analogues with insulin analogues for treatment of diabetes.

### BACKGROUND

Diabetes mellitus, or simply diabetes, is a metabolic condition, or combination of conditions, in which an individual displays high concentrations of blood glucose. The condition is caused either by insufficient production of insulin within the body or by the failure of cells to respond properly to the insulin that is produced.

Diabetes is one of the leading causes of death and disability in the United States, affecting approximately 8 percent of the United States population, with total cost of diabetes in the United States alone is estimated to approach $200 billion and in other developed countries. There are over 422 million people living with diabetes worldwide; 5-10% of these people have type 1 diabetes. Diabetes is associated with long-term complications affecting almost every aspect of the body. Patients often suffer from severe side effects such as obesity, heart disease, hypertension, stroke, bone and joint problems, and circulatory disruptions leading to kidney failure, vision loss, nerve damage, infections, and limb amputation, most, if not all of which, are preventable with tight glycemic control. Diabetes has also been associated with depression and dementia.

Type 1 diabetes occurs after an autoimmune response resulting in the destruction of pancreatic β cells responsible for production and secretion of metabolically active hormones including insulin and amylin. Patients with type 1 diabetes cannot produce the insulin required for glucose uptake by cells. Symptoms of type 1 diabetes include increased thirst and urination, hunger, weight loss, blurred vision, and extreme fatigue. Although it can appear at any age, type 1 diabetes most frequently develops in children and young adults. Amylin complements the action of insulin to regulate blood glucose levels by acting centrally to slow gastric emptying, suppress postprandial glucagon secretion, and decrease food intake by increasing satiety. Similar to insulin, amylin production is completely absent in individuals with type 1 diabetes.

Type 2 diabetes is the most common form of diabetes, accounting for 90 to 95 percent of all cases of diabetes. Type 2 diabetes is generally associated with older age, obesity, family history, previous history with gestational diabetes, and physical inactivity. It also tends to be more prevalent in certain ethnicities. Type 2 diabetes is also referred to as insulin-resistant diabetes because the pancreas is usually able to produce sufficient amounts of insulin, but the body fails to respond properly to that insulin. As with type 1 diabetes, blood glucose levels in individuals suffering from type 2 diabetes increase, and the body is unable to metabolize the blood glucose efficiently. The symptoms of type 2 diabetes generally develop more slowly than those of type 1 diabetes. The symptoms include fatigue, frequent urination, increased thirst and hunger, weight loss, blurred vision, and slow healing of wounds or sores. In some cases, no symptoms are evident.

Gestational diabetes occurs in approximately 3 to 8 percent of pregnant women in the United States, generally developing late in pregnancy. The disease typically disappears after birth of the baby, but women who have experienced gestational diabetes are significantly more likely to develop type 2 diabetes within 5 to 10 years than those who have not. Women who maintain reasonable body weight and are physically active after suffering from gestational diabetes may be less likely to develop type 2 diabetes than those who do not. As with type 2 diabetes, gestational diabetes occurs more frequently among women with a family history of diabetes and also in certain ethnic groups.

Since the discovery of insulin over 80 years ago, diabetes, particularly type 1, or insulin-dependent diabetes, has been a somewhat treatable condition. The combination of a proper diet, physical activity, and insulin injection, together with the monitoring of blood glucose levels using portable meters, allows the management of type 1 diabetes. For type 2 diabetics, healthy eating, physical activity, and monitoring blood glucose levels are also important. In some cases, drug therapies can be used to control blood glucose levels in these patients.

Insulin replacement therapy has been the focus of diabetes treatment for almost 100 years. Current treatments use subcutaneous injections or infusion from pumps to deliver insulin. On the other hand, amylin, which is critical to regain suppression of post-prandial glucagon, which cannot be achieved with subcutaneous insulin alone, has largely been overlooked. A true hormone replacement therapy for patients with type 1 diabetes would ideally simultaneously deliver amylin and insulin. Treatment of diabetes with a combination of insulin and amylin analogues is more effective than insulin alone. However, amylin replacement therapy has proven to be challenging because amylin is highly unstable in formulation and rapidly aggregates into amyloid fibrils, prompting the development of the amylin analogue pramlintide, which acts through similar mechanisms to amylin in vivo. Further adding to the challenge, amylin analogues, including pramlintide, are incompatible with insulin analogues (e.g., aspart, lispro, glulisine) in standard formulations. Pramlintide differs from amylin by alterations to three amino acids that suppress amyloid fibrillation and enable its stable formulation at pH˜4. Unfortunately, insulin and its analogues are typically formulated at pH˜7.4. Current treatments require separate subcutaneous administrations, increasing patient burden and limiting pramlintide adoption to less than 1.5% of rapid-acting insulin users. Furthermore, the minimal overlap between the pharmacokinetics of these two hormones following administration limits the potential synergistic effects of the dual-hormone treatment. Notwithstanding these challenges, patients treated with a combination of insulin and pramlintide at mealtimes have exhibited improved glycemic control compared with patients treated with insulin alone. Despite the increased efficacy of dual-hormone treatment, by 2012 only 29,000 patients, of over 2,100,000 patients who would potentially benefit from such a treatment, had adopted it due to the burdensome requirement for administration in two separate injections.

In addition to formulation challenges, the pharmacokinetics of insulin and pramlintide in current formulations are highly dissimilar and the resulting lack of pharmacokinetic overlap does not mimic their natural mode of action. In non-diabetic people, insulin and amylin are co-secreted at a fixed ratio from the β-cells in the pancreas and act with similar kinetics. In contrast, current “rapid-acting” insulin analogue formulations HUMALOG® (insulin lispro) and NOVOLOG® (insulin aspart) exhibit delayed onset of action of ˜20-30 min, peak action at ˜60-90 min and total duration of action of ˜3-4 hours, while SYMLIN® (pramlintide) begins to act almost immediately, exhibits peak action at ˜20 min and total duration of action of ˜90 min. This large dissimilarity in pharmacokinetics arises from the distinct aggregation states of the proteins in formulation and the resulting impact on absorption behavior. These insulin formulations contain a mixture of hexamers, dimers and monomers, which, upon subcutaneous injection, dissociate and are absorbed at different rates resulting in the delayed onset and long duration of action of these formulations (FIG. 1B, left side). In contrast, the pramlintide monomer is absorbed rapidly from the subcutaneous space (FIG. 1B, right side). The lack of overlap between insulin and pramlintide pharmacokinetics in current treatment strategies hinders the synergistic effects of pramlintide and insulin action. Recent clinical studies are moving towards evaluating the benefits of delivering a fixed ratio of insulin and pramlintide using two separate pumps to better simulate endogenous insulin-pramlintide secretion. While the use of two separate pumps can deliver a fixed ratio of pramlintide with insulin, this method is overly burdensome outside of a research setting and does not address the poor pharmacokinetic overlap of these two hormones following subcutaneous administration.

A new class of excipients is needed for protein formulation to address concerns surrounding aggregation and denaturation over time. Covalent PEGylation has been successful as a strategy to stabilize insulin and amylin in formulation; however, covalent modification of proteins often interferes with their activity, typically extends their pharmacokinetics in vivo, and can lead to increased immunogenicity. Recent research has shown that non-covalent modification of proteins can enhance their stability in formulation. In particular, cucurbit[n]urils (CB[n]) are a family of macrocyclic hosts that exhibit strong binding affinities for aromatic amino acids, and have a reassuring safety profile. Conjugation of a polyethylene glycol (PEG) chain to CB[7] creates a designer excipient (CB[7]-PEG) for non-covalent PEGylation of protein therapeutics. Insulin has an N-terminal phenylalanine and pramlintide has an amidated C-terminal tyrosine, making them ideal targets for supramolecular modification using the CB[7]-PEG platform.

The need remains for an insulin-pramlintide co-formulation with increased pharmacokinetic overlap and lower treatment burden to enable a strategy for glycemic control in treatment of diabetes that strongly mimics the endogenous mechanism of co-excretion of insulin and amylin in the pancreas.

### BRIEF SUMMARY

In one aspect of the invention, a method for co-formulation of insulin-pramlintide co-formulation employs simultaneous supramolecular PEGylation of insulin and pramlintide. The inventive approach exploits strong and specific host-guest interactions of cucurbit[7]uril-PEG with end-terminal aromatic amino acids on the proteins. This dual-hormone co-formulation is stable for over 100 hours under stressed conditions, compared to only 10 hours for commercial insulin formulations. Using a rat model of insulin-deficient diabetes, co-formulation simultaneously is shown to modify the pharmacokinetic profiles of insulin and pramlintide, increasing overlap from 40% to 70% when compared to administration in separate injections. This approach to insulin-pramlintide co-formulation more closely mimics endogenous co-secretion of insulin and amylin and shows promise as a dual-hormone replacement therapy for the treatment of diabetes that both enhances the efficacy and reduces the burden of treatment.

The present invention is based on the discovery of a method for co-formulation and stabilization of amylin analogues with insulin analogues in the presence of a cucurbit[7]uril (CB[7])-poly(ethylene glycol) (PEG) conjugate for use in treating diabetes. The co-formulation simplifies administration by allowing amylin analogues and insulin analogues to be administered together in a single injection. Moreover, bioavailability and pharmacokinetics as well as safety and efficacy are improved by the co-formulation.

In one aspect, a pharmaceutical composition comprises a) amylin or an amylin analogue; b) insulin or an insulin analogue; and c) a CB[7]-PEG conjugate in an effective amount sufficient to inhibit formation of amyloid fibrils. The amylin analogue may be pramlintide and the insulin analogue may be insulin aspart or insulin lispro. In some embodiments, the CB[7]-PEG prevents protein precipitation for at least 100 hours. The insulin or insulin analogue is preferably zinc free, and may be formulated with ethylenediaminetetraacetic acid (EDTA) to remove formulation zinc. Each of the amylin or amylin analogue and the insulin or insulin analog have similar hydrodynamic sizes.

In another aspect of the invention, a pharmaceutical composition comprises a co-formulation formed by simultaneous supramolecular PEGylation at physiological pH of amylin or an amylin analogue and insulin or an insulin analogue with CB[7]-PEG in the absence of formulation zinc. The amylin analogue may be pramlintide and the insulin analogue may be insulin aspart or insulin lispro. In some embodiments, the CB[7]-PEG prevents protein precipitation for at least 100 hours. The insulin or insulin analogue is preferably zinc free, and may be formulated with ethylenediaminetetraacetic acid (EDTA) to remove formulation zinc. Each of the amylin or amylin analogue and the insulin or insulin analog have similar hydrodynamic sizes.

In another aspect, of the invention, a pharmaceutical composition comprises amylin or an amylin analogue and a CB[7]-PEG conjugate. In some embodiments, the CB[7]-PEG conjugate is in an effective amount sufficient to inhibit formation of amyloid fibrils.

In yet another aspect, a method of treating a subject for diabetes includes administering a therapeutically effective amount of a pharmaceutical composition including amylin or an amylin analogue; insulin or an insulin analogue; and a CB[7]-PEG conjugate in an effective amount to the subject.

In still another aspect, a kit includes a pharmaceutical composition described herein and instructions for treating type 1 or type 2 diabetes. The kit may further comprise means for delivering the pharmaceutical composition to a subject.

### BRIEF DESCRIPTION OF THE DRAWINGS

FIGS. 1A-1G illustrate how CB[7]-PEG binds to insulin and pramlintide and alters diffusion rates in formulation. FIG. 1A diagrammatically illustrates a comparison of post-mealtime metabolic signaling pathways in non-diabetic people and type 1 diabetic people receiving insulin replacement therapy; FIGS. 1B and 1C diagrammatically illustrate how molecular weight affects diffusion rates, which directly impacts absorption kinetics following subcutaneous insulin administration for standard insulin formulations and after complexation with CB[7]-PEG, respectively; FIGS. 1D and 1F are plots indicating binding of CB[7] to both proteins in acridine orange competitive binding assays of aspart and pramlinitide, respectively; FIGS. 1E and 1G are diffusion-ordered NMR Spectroscopy (DOSY) plots providing insight into the formation of protein/CB[7]-PEG complexes and their rates of diffusion in formulation; FIGS. 1H and 1I show circular dichroism spectra from 200-260 nm for aspart and pramlintide, respectively; FIG. 1J provides 1H NMR results demonstrating Insulin/CB[7]-PEG binding for insulin, insulin+free PEG5k, CB[7]-PEG, and insulin/CB[7]-PEG complex; FIG. 1L provides 1H NMR titration results demonstrating insulin/CB[7]-PEG binding, where insulin/CB[7]-PEG complex can be tracked by the emergence of the characteristic peak ˜6.4 ppm.

FIGS. 2A-2C illustrate that formulation with CB[7]-PEG stabilizes a co-formulation of pramlintide, NOVOLOG® and HUMALOG®, respectively, at pH˜7.4; FIGS. 2D and 2E provide additional results for NOVOLOG® and HUMALOG®, respectively, with EDTA as a chelator.

FIGS. 3A-3O illustrate the pharmacokinetics of aspart and pramlintide following different administration routes in diabetic rats, where FIGS. 3A-3C show blood glucose levels at ratios of 1:15, 1:8 and 1:2 aspart to pramlintide, respectively; FIGS. 3D and 3F show pharmacokinetics of insulin aspart and pramlintide, respectively; FIGS. 3E and 3G show the area under the pharmacokinetic curves of aspart (p=0.0012) and pramlintide for the first 60 or 40 minutes, respectively, after subcutaneous injection. FIGS. 3H and 3L plot the normalized serum concentration for aspart and pramlintide, respectively. FIGS. 3I and 3M show the time to reach 50% of peak aspart or pramlintide serum concentration, respectively. FIGS. 3J and 3N plot the time to reach peak serum concentrations, and FIGS. 3K and 3O plot time for depletion to 50% of peak serum concentration for aspart and pramlintide, respectively.

FIGS. 4A-4B are plots of mean normalized serum concentration (normalized for each individual rat) of NOVOLOG® and Pramlintide when administered as two separate injections (FIG. 4A) or pramlintide-aspart co-formulation with CB[7]-PEG at physiologic pH (FIG. 4B). FIG. 4C provides the ratio of the area under the curve (AUC) of the pharmacokinetic profiles of pramlintide and aspart for administration as separate injections and as a co-formulation.

FIGS. 5A-5I show the pharmacokinetics of insulin lispro in mU/L (FIG. 5A) or c, pramlintide in pM (FIG. 5C). FIGS. 5B and 5D plot the AUC of lispro (*p=0.017) and, pramlintide, respectively, for the first 240 minutes after subcutaneous injection. Pharmacokinetics for each pig were individually normalized to peak concentrations and normalized values were averaged for lispro (FIG. 5E) or pramlintide (FIG. 5I) concentration for each treatment group. FIGS. 5F and 5J plot time to reach 50% of peak for lispro and pramlintide, respectively, (*p=0.018) concentration (onset). FIGS. 5G and 5K plot time to reach peak lispro and pramlintide concentration. respectively. FIGS. 5H and 5L show time for lispro and pramlintide, respectively, (*p=0.019) to depletion of 50% of peak concentration. Error bars, mean±s.d., n=12-15 for all groups. The Grubbs' outlier test (alpha=0.05) was used to remove outliers. Statistical significance was determined by a two-tailed student's t-test.

FIGS. 6A-6B plot mean normalized concentration of lispro and pramlintide when administered as two separate injections and as a co-formulation with CB[7]-PEG, respectively. FIG. 6C shows the overlap between curves as the time during which both lispro and pramlintide concentrations were greater than 0.5 (width at half peak height), shown as a ratio of c, overlap time over the total width of both peaks (Overlap/(Lispro+Pramlintide−Overlap)). FIG. 6D plots change in glucagon concentrations from baseline over 4-hours following treatment administration. FIG. 6E plots the overall distance from baseline by treatment group (sum of individual points). FIG. 6F is a summary schematic of how treatment affects post-prandial glucagon.

FIGS. 7A-7F are blood chemistry panels in rats for, respectively, ALT, AST, ALP, bilirubin, BUM, and creatinine, performed to evaluate biocompatibility of CB[7]-PEG; FIGS. 7G and 7H are histology sections taken at 40× and 200× magnification, respectively.

FIG. 8 provide measured blood chemistry values in pigs for AST, ALT, BUN, bilirubin and creatinine.

### DETAILED DESCRIPTION OF EMBODIMENTS OF THE INVENTION

The practice of the present invention will employ, unless otherwise indicated, conventional methods of medicine, pharmacology, chemistry, biochemistry, and molecular biology and recombinant DNA techniques within the skill of the art. Such techniques are explained fully in the literature. See, e.g., J. Unger Diabetes Management in Primary Care (LWW; second edition, 2012); Practical Insulin: A Handbook for Prescribing Providers (American Diabetes Association; 4th edition, 2015); A. Young Amylin, Volume 52: Physiology and Pharmacology (Advances in Pharmacology, Academic Press, 2005); Handbook of Experimental Immunology, Vols. I-IV (D. M. Weir and C. C. Blackwell eds., Blackwell Scientific Publications); A. L. Lehninger, Biochemistry (Worth Publishers, Inc., current addition);); Sambrook et al., Molecular Cloning: A Laboratory Manual (Cold Spring Harbor Laboratory Press, 3rd Edition, 2001); Methods In Enzymology (S. Colowick and N. Kaplan eds., Academic Press, Inc.).

All publications, patents and patent applications cited herein, whether supra or infra, are hereby incorporated by reference in their entireties.

### Definitions

In describing the present invention, the following terms will be employed, and are intended to be defined as indicated below.

The term “analogue” refers to biologically active derivatives of the reference molecule that retain desired activity, such as insulin or amylin activity for use in the treatment of type 1 or type 2 diabetes as described herein. In general, the term “analogue” refers to compounds having a native polypeptide sequence and structure with one or more amino acid additions, substitutions (generally conservative in nature) and/or deletions, relative to the native molecule, so long as the modifications do not destroy biological activity and which are “substantially homologous” to the reference molecule as defined below. In general, the amino acid sequences of such analogues will have a high degree of sequence homology to the reference sequence, e.g., amino acid sequence homology of more than 50%, generally more than 60%-70%, even more particularly 80%-85% or more, such as at least 90%-95% or more, when the two sequences are aligned. Methods for making polypeptide analogues are known in the art and are described further below.

The term “derivative” is intended to mean any suitable modification of the native polypeptide of interest, of a fragment of the native polypeptide, or of their respective analogues, such as glycosylation, phosphorylation, polymer conjugation (such as with polyethylene glycol), or other addition of foreign moieties, as long as the desired biological activity of the native polypeptide is retained. Methods for making polypeptide fragments, analogues, and derivatives are generally available in the art.

The term “subject” is meant any member of the subphylum chordata, including, without limitation, humans and other primates, including non-human primates, mammals, and birds.

The term “physiologic pH” or “physiological pH” means the pH that normally prevails in the body. For humans, physiologic pH is within a range of 7.35-7.45, typically around 7.4.

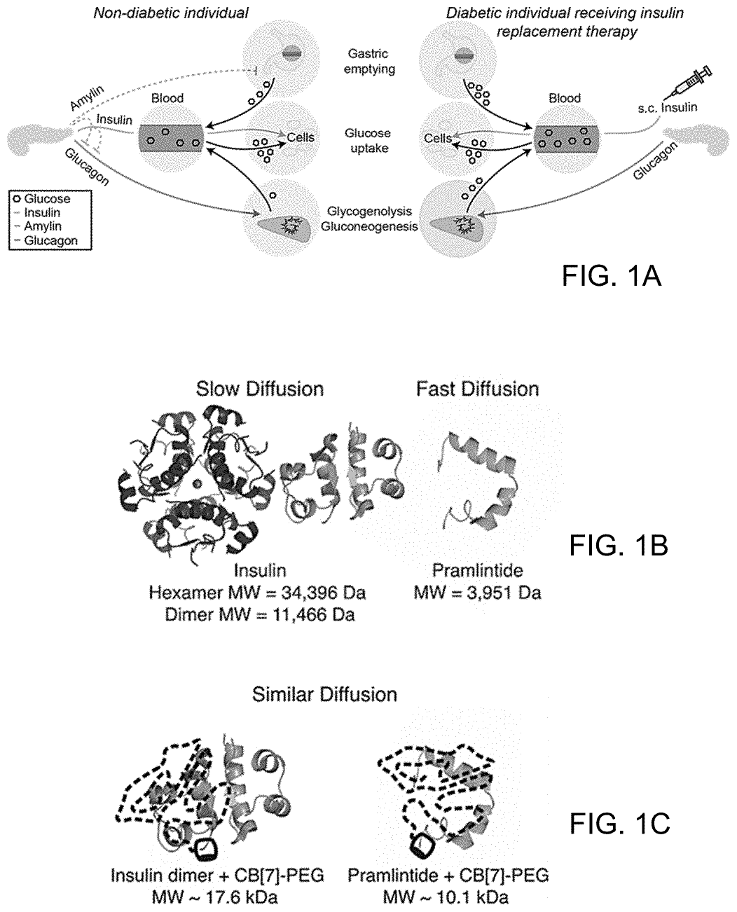

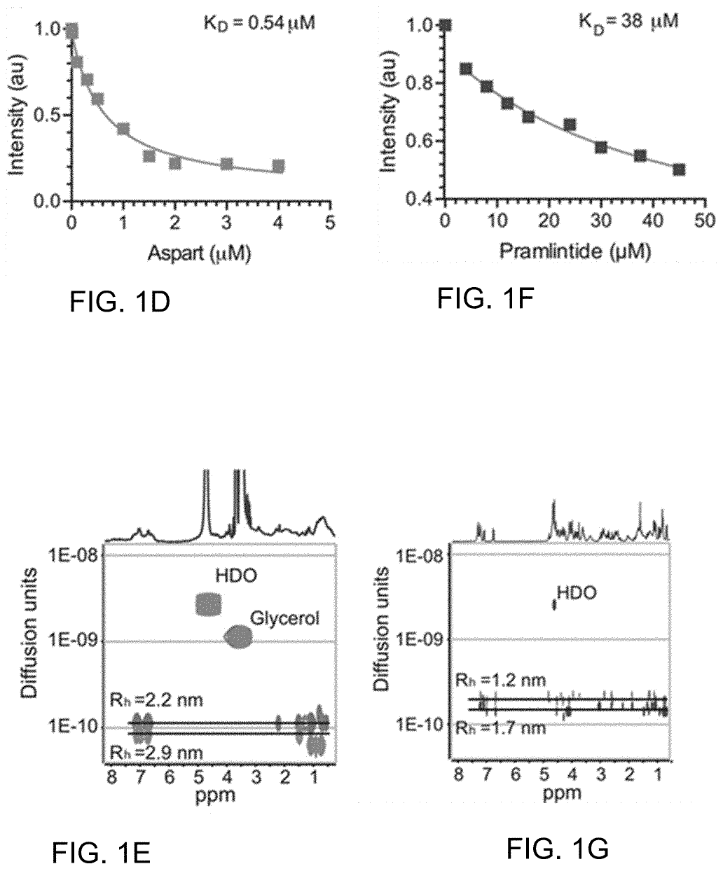

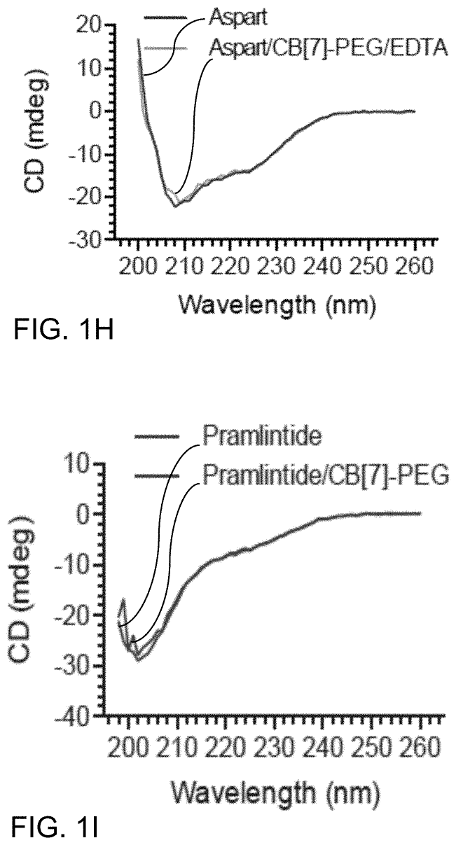

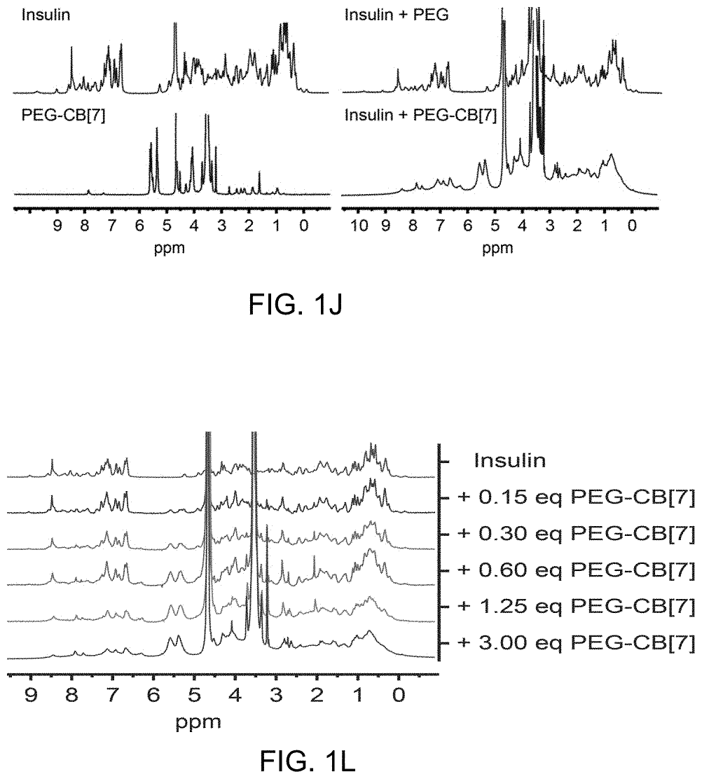

FIG. 1A illustrates a scheme of post-mealtime metabolic signaling pathways in non-diabetic people and type 1 diabetic people receiving insulin replacement therapy. In non-diabetic people (left side), endogenous insulin promotes cellular glucose uptake and acts with amylin to locally suppress post-prandial glucagon, thus decreasing glycogenolysis & gluconeogenesis. In contrast, as shown on the right side of FIG. 1A, treatment of diabetic patients with subcutaneous (“s.c.”) insulin alone cannot restore glucagon suppression. Amylin replacement is critical to fully restore metabolic signaling and constitute a true hormone replacement therapy.

The present invention is based on the discovery that amylin analogues can be co-formulated with insulin analogues in the presence of CB[7]-PEG, which stabilizes the proteins and inhibits their aggregation into amyloid fibrils. The following detailed description provides examples of methods for preparation of pharmaceutical compositions comprising co-formulations of amylin analogues and insulin analogues and methods of using such pharmaceutical compositions for treatment of type 1 and type 2 diabetes.

CB[7]-PEG with varying PEG molecular weights has been shown to bind to recombinant human insulin with micromolar affinities, increasing its stability in formulation and enabling simple tuning of the duration of insulin action in a mouse model of insulin-deficient diabetes through modulation of the PEG molecular weight. The present invention exploits CB[7]-PEG for simultaneous supramolecular PEGylation of insulin and pramlintide to stabilize the two hormones in a co-formulation whereby the optimal therapeutic ratio is defined in the formulation. This dual hormone therapy can be administered in a single injection, thus reducing burden on the subject and/or their caregivers. As a further benefit, the increased overlap of the pharmacokinetics of the two drugs enhances their efficacy in diabetes management.

In some embodiments, the protein, i.e., the amylin or amylin analogue and/or the insulin or insulin analog, in the pharmaceutical composition will be stable for at least 15 hours, at least 60 hours, or at least 100 hours, at least 2, 3, 4, or 5 weeks, at least 1, 2, 3, 4, 5, 6, 7, 8, 9, 10, 11, or at least 12 months. The stability of the protein in the composition may be measured by the change in transmittance at 540 nm, e.g., as described in Example 2 below. The protein will preferably exhibit no more than a 5% change, and more preferably no more than 2% change, in transmittance at 540 nm for at least 15 hours, at least 60 hours, or at least 100 hours, at least 2, 3, 4, or 5 weeks, at least 1, 2, 3, 4, 5, 6, 7, 8, 9, 10, 11, or at least 12 months. The protein is preferably stable for at least 15 hours, more preferably stable for at least 60 hours, and even more preferably stable for at least 100 hours.

As disclosed herein, administering a pharmaceutical composition by a single injection of the co-formulation results in improved suppression of post-prandial glucagon levels as compared to separate administrations of the amylin or amylin analogue and the insulin or insulin analogue. More specifically, administering the co-formulation results in suppression of post-prandial glucagon levels by at least 30%, at least 50%, at least 80%, or at least 90% as compared to separate administrations of the amylin or amylin analogue and the insulin or insulin analogue. The suppression of post-prandial glucagon levels may be measured by an assay as described in Example 5 below.

In some embodiments, the molar ratio of CB[7]-PEG to the insulin or insulin analogue is from about 1:1 to about 10:1, and more specifically from about 3:1 to about 5:1. The molar ratio of CB[7]-PEG to the insulin or insulin analogue may be at least about 3:1 or at least about 5:1. The inventive pharmaceutical composition may comprise the insulin or insulin analogue at a concentration of from about 50 U/mL to about 200 U/mL and may be about 100 U/mL.

The molar ratio of the amylin or amylin analogue to the insulin or insulin analogue may be from about 1:1 to about 1:20, specifically about 1:2 to about 1:15, and more specifically, about 1:2 to about 1:6. In some embodiments, the molar ratio of the amylin or amylin analogue to the insulin or insulin analogue may be in a range of about 1:6 to about 1:8 or a range of about 1:8 to about 1:15. In other embodiments, the molar ratio of the amylin or amylin analogue to the insulin or insulin analogue may be about 1:2, about 1:6, about 1:8, or about 1:15.

### EXAMPLES

The following examples are offered for illustrative purposes only, and are not intended to limit the scope of the present invention.

### Example 1: Characterization of Insulin and Pramlintide Binding to CB[7]-PEG

CB[7]-PEG was prepared according to published protocols including those described by Webber, M. J. et al., “Supramolecular PEGylation of biopharmaceuticals”, P. Natl. Acad. Sci. USA. 113, 14189-14194 (2016), and US. Pat. Publ. No. US2018/0296680 of Webber, et al., which are incorporated herein by reference. NOVOLOG® (Novo Nordisk®, Bagsværd, Denmark) and pramlintide (BioTang) were purchased and used as received. All other reagents were purchased from Sigma-Aldrich unless otherwise specified.

Previous studies by Webber et al. observed increased stability of recombinant human insulin with the addition of CB[7]-PEG₅ₖ, CB[7]-PEG₁₀ₖ, and CB[7]-PEG₃₀ₖ and showed that increased PEG chain length prolongs the effect of insulin on blood glucose levels in a mouse model of insulin-deficient diabetes. To demonstrate the inventive approach, CB[7]-PEG₅ₖ was employed due to its demonstrated capacity to stabilize recombinant human insulin in formulation without significantly extending insulin duration of action in vivo. The goal was to not only stabilize insulin and pramlintide together in formulation, but to also use co-formulation as an opportunity to simultaneously alter the pharmacokinetics of the two hormones in vivo to more closely match one another. Through a combination of insulin hexamer disruption by removal of formulation zinc and simultaneous complexation of insulin and pramlintide with CB[7]-PEG, the effective hydrodynamic size of both components become similar to one another, as shown diagrammatically in FIGS. 1B and 1C. Without wishing to be bound by a particular theory, it is believed that this similarity in hydrodynamic size, which directly impacts absorption following subcutaneous administration, promotes greater overlap between the pharmacokinetic profiles of the two therapeutics.

For these studies, unmodified CB[7] was purchased from Strem Chemicals and Acridine Orange (AO) was purchased from Sigma-Aldrich. Binding of CB[7] to NOVOLOG® and pramlintide was assessed using the AO dye displacement assay, as previously described. Briefly, 6 μM of CB[7] and 8 μM AO (for NOVOLOG® assay) or 2 μM AO (for pramlintide assay) were combined with 100 μL of either NOVOLOG® or Pramlintide samples. NOVOLOG® samples were diluted to concentrations of 0, 0.01, 0.1, 0.3, 0.5, 1, 1.5, 2, 3, 4 μM in H₂O. Pramlintide samples were diluted to concentrations of 0, 4, 8, 12, 18, 24, 30, 37.5, 40 μM in H₂O. Samples were incubated overnight in light-free conditions, and fluorescent spectra were collected on an BioTek SynergyH1 microplate reader, exciting at 485 nm and collecting the resulting fluorescent spectra from 495 to 650 nm. The decay in the peak of AO fluorescent signal was fit to a one-site competitive binding model (GraphPad Prism, version 6.0), using the CB[7]⋅AO equilibrium constant reported previously (K_{eq}=2×10⁵ M⁻¹), to determine binding constants of unmodified CB[7] to insulin and pramlintide.

Webber et al. previously confirmed that the presence of PEG in the CB[7]-PEG conjugate does not alter the binding affinity of CB[7] to insulin. As shown in FIG. 1D, the equilibrium dissociation constant for CB[7] to aspart was determined to be 0.54 μM, which is similar to values previously reported for binding to recombinant insulin. Pramlintide binding affinity to CB[7] was determined to be 38 μM, as seen in FIG. 1F. The higher binding affinity of CB[7] to insulin compared to pramlintide is due to the well-known increase in binding affinity when a hydrophobic guest is flanked by a protonated amine group. Circular dichroism confirmed that binding of both aspart and pramlintide with CB[7]-PEG did not affect protein structure, as shown in FIGS. 1H and 1I.

As is known in the art, molecular weight affects the rate at which compounds can traffic to the lymphatic circulation. Thus, supramolecular PEGylation using PEG chains of various molecular weights may contribute to delayed and controlled uptake, creating a sustained source of insulin in the s.c. space by increasing the effective molecular weight of the complex as a result of CB[7]-PEG binding. In some embodiments, the PEG molecule in CB7-PEG may have a molecular weight less than or about 1 kDa, facilitating preferential absorption via capillary circulation. In other embodiments, the PEG may have a molecular weight of from 1-5 kDA or from 1-10 kDa, and may be about 5 kDa or about 10 kDa. In still other embodiments, the PEG will have a molecular weight within the range of 10-30 kDa, and may be approximately 30 kDa. In other embodiments, the PEG may have a molecular weight greater than 30 kDa and less than about 100 kDa.

Having confirmed that CB[7]-PEG binds to aspart and pramlintide, diffusion-ordered NMR spectroscopy (DOSY) was used to provide insight into the size and diffusion characteristics of the protein/CB[7]-PEG complexes (FIGS. 1E, 1G). ¹H 2D DOSY spectra were recorded at a protein concentration of 6 mg mL⁻¹ in 200 mM phosphate buffer, pH 7, in D₂O. 1D H¹-NMR of the complex showed a broadening of both insulin and CB[7]-PEG signals (FIG. 1J). This was exacerbated with an increasing ratio of CB[7]-PEG to insulin, as shown in FIG. 1L). As such, an optimum ratio of CB[7]-PEG to insulin for DOSY was established to 1.25 mol. A Varian Inova 600 MHz NMR instrument was used to acquire the data. Magnetic field strengths ranging from 2 to 57 G cm⁻¹. The DOSY time and gradient pulse were set at 132 ms (Δ) and 3 ms (δ) respectively. All NMR data were processed using MestReNova 11.0.4 software.

In these tests, aspart was formulated with CB[7]-PEG and ethylenediaminetetraacetic acid (EDTA) to remove formulation zinc. EDTA forms strong complexes with zinc (KD ˜10×10−18 M) and addition of one molar equivalent of EDTA relative to the zinc ion in insulin formulations rapidly sequesters the zinc, preventing it from interacting with the insulin and thus disrupting the insulin hexamer in solution. In DOSY experiments, CB[7]-PEG and aspart were found to diffuse together, verifying the binding interaction observed previously using competitive binding assays. The aspart dimer exhibited a diffusion rate of D˜1.2×10⁻¹⁰ m² s⁻¹, while the complex of aspart/CB[7]-PEG exhibited a 30% lower diffusion rate of D˜8.7×10⁻¹¹ m² s⁻¹. The Stokes-Einstein relationship specifies that the diffusion rate, D, is inversely proportional to the size of the species in solution, whereby a 50% increase in the molecular weight is expected to decrease the diffusion rate by roughly ⅓, as observed in this study. This relationship was used to approximate the hydrodynamic radius (Rh) to be 2.2 nm for dimeric aspart and 2.9 nm for the aspart/CB[7]-PEG complex. For comparison, the insulin hexamer has a hydrodynamic radius of approximately 2.8 nm. Similar to aspart, the diffusion rate decreases from D˜2×10⁻¹⁰ m² s⁻¹ for pramlintide alone to D˜1.4×10⁻¹⁰ m² s⁻¹ for the pramlintide/CB[7]-PEG complex, corresponding to a change in Rh from 1.2 nm to 1.7 nm. The degree of diffusion rate increase after the addition of CB[7]-PEG to pramlintide is less than that observed for aspart likely on account of the weaker and more dynamic binding. We observed that the ratio of the diffusion rates for the pramlintide/CB[7]-PEG and aspart/CB[7]-PEG complexes is ˜1.6, while the ratio of diffusion rates for pramlintide alone and insulin in a standard formulation is ˜2.3, indicating that the formulation of the two hormones with CB[7]-PEG without zinc creates supramolecular protein-PEG complexes that are much more similar in size than is possible with standard formulation approaches.

To confirm that CB[7]-PEG binding did not change the secondary structure of insulin aspart or pramlintide, we used circular dichroism to characterize the secondary protein structures when bound to CB[7]-PEG (FIG. 1E). Novolog® was diluted to 0.2 mg/mL in PBS (pH=7.4) and was evaluated (i) alone, (ii) with EDTA at a 1:1 molar ratio to zinc, (iii) with CB[7]-PEG at a 5:1 molar excess to insulin (1.1 mg/mL), and (iv) with both EDTA and CB[7]-PEG. The concentration of CB[7]-PEG in formulation results in 93% of insulin bound. Pramlintide was evaluated (i) alone in PBS at 0.5 mg/mL and (ii) with an excess of CB[7]-PEG at a concentration of 1.1 mg/mL. After mixing, samples were left to equilibrate for 15 minutes at room temperature. Near-UV circular dichroism spectroscopy was performed at 20° C. with a J-815 CD Spectropolarimeter (Jasco Corporation) over a wavelength range of 185-250 nm using a 0.1 cm pathlength cell.

Aspart was formulated with CB[7]-PEG (0.2 mM) to ensure that greater than 95% of the protein would be complexed. CB[7]-PEG did not alter the secondary structure of either aspart or pramlintide in formulation, whether in the presence or absence of EDTA to sequester formulation zinc (FIG. 1H). Under the same formulation conditions, as shown in FIG. 1I, pramlintide was approximately 80% bound and no difference in protein secondary structure was observed. From this evaluation, we concluded that formulation with CB[7]-PEG does not significantly alter the structure of insulin and pramlintide.

### Example 2: In Vitro Stability

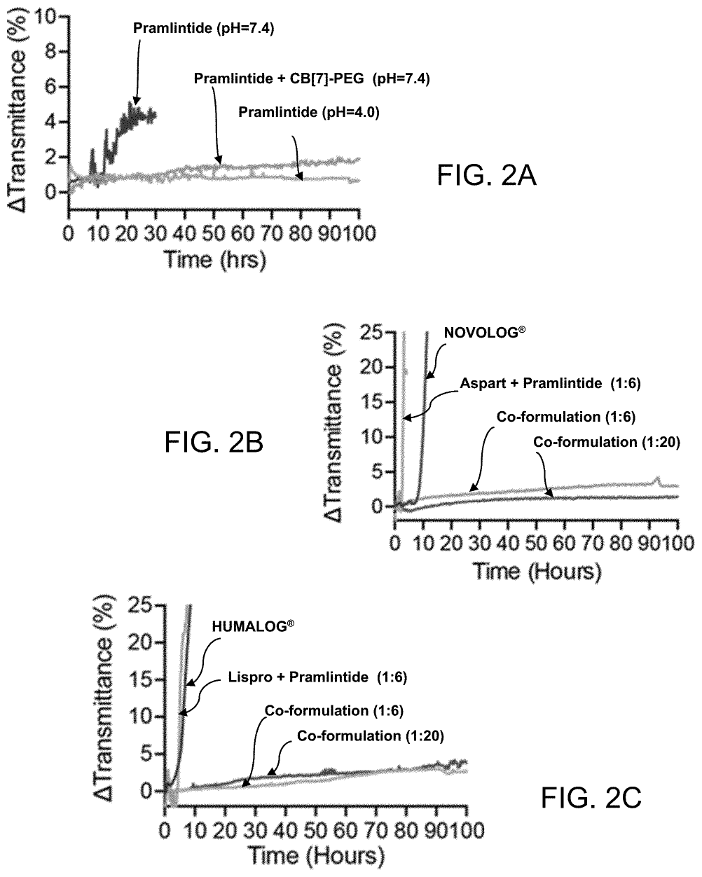

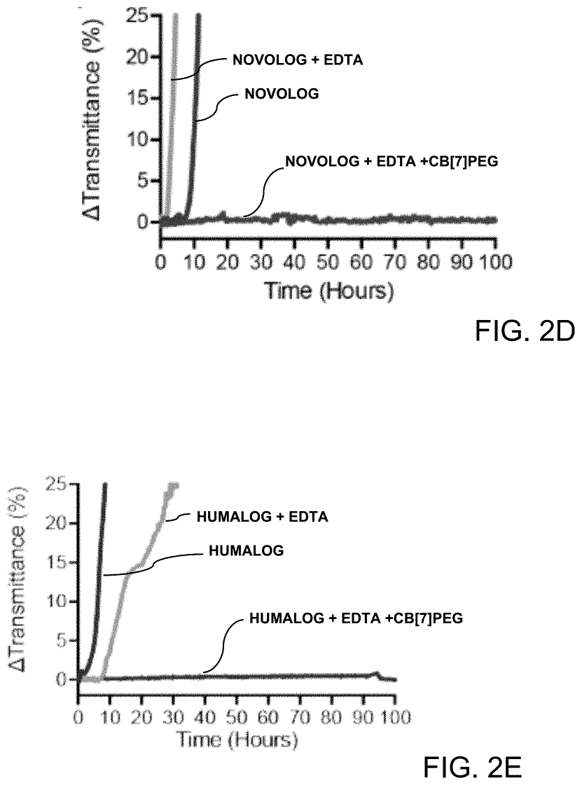

To determine if CB[7]-PEG stabilized pramlintide in combination with insulin at physiological pH, insulin and pramlintide aggregation was assessed under stressed conditions (37° C. with continuous agitation). As insulin and pramlintide destabilize, they form amyloid fibrils that are insoluble, inactive, and often immunogenic. These aggregates are large and scatter light, allowing the degree of aggregation to be evaluated by measuring the change in transmittance over time. Kinetic profiling of the aggregation of insulin formulations was performed using change in transmittance at 540 nm, a wavelength at which both insulin and the CB[7]-PEG have negligible absorbance. Experiments were conducted at pH˜7.4, 37° C., in physiological buffer with continuous agitation over the course of 100 h, demonstrating that formulation with CB[7]-PEG resists aggregation over the period assayed. Data shown in FIGS. 2A-2E are the average transmittance trace for n=3 samples per group.

Methods for aggregation assays for recombinant human insulin were adapted from Webber et al. Briefly, samples were plated at 150 μL per well (n=3/group) in a clear 96-well plate and sealed with optically clear and thermally stable seal (VWR). The plate was immediately placed into an plate reader and incubated with continuous shaking 37° C. Absorbance readings were taken every 10 minutes at 540 nm for 100 h (BioTek SynergyH1 microplate reader). The aggregation of insulin leads to light scattering, which results in reduction of sample transmittance. Aggregation time (tA), according to Webber et al., is the time after which a 10% reduction in transmittance is observed—native insulin aggregates after 13.6±0.2 h of agitation and insulin formulated with unmodified CB[7] displays an aggregation time of 14.2±0.4 h. In FIGS. 2A-2E, time (hours) is plotted against the percentage change in transmittance (“Δ Transmittance”) from the transmittance at time zero, with a <10% change being defined as “aggregation” in insulin or insulin analogues, or their co-formulations.

Controls included: (i) NOVOLOG®, (ii) zinc-free NOVOLOG®, (iii) pramlintide (sodium acetate buffer at pH=4), (iv) pramlintide (PBS at pH=7), (v) NOVOLOG®+pramlintide (PBS at pH=7.4). Zinc(II) was removed from the insulin through competitive binding by addition of ethylenediaminetetraacetic acid (EDTA), which exhibits a dissociation binding constant approaching attomolar concentrations (KD˜10-18 M). EDTA was added to formulations (1:1 molar ratio to zinc) to sequester zinc from the formulation. The stability of formulations mixed with CB[7]-PEG evaluated were: (i) zinc-free aspart (100 U/mL)+CB[7]-PEG (5:1 molar excess to insulin), (vii) pramlintide (PBS at pH=7)+CB[7]-PEG (5:1), (viii) zinc-free aspart+pramlintide+CB[7]-PEG (5 eq).

In these assays, the results of which are shown in FIGS. 2D and 2E, commercial NOVOLOG® (aspart) and HUMALOG® (lisporo) aggregated in 10±1.0 hrs (n=3) and 6±0.2 hrs, respectively, under stressed conditions while corresponding zinc-free formulations (created using a 1:1 molar ratio of EDTA to zinc) were significantly less stable and aggregated following only 3.2±0.2 hrs (n=3). These observations suggest that removal of formulation zinc destabilizes the insulin hexamer and encourages insulin aggregation, consistent with previous work. In contrast, neither NOVOLOG® nor HUMALOG® with the addition of CB[7]-PEG and zinc-free aspart/CB[7]-PEG formulations aggregated during the entire 100-hour kinetic testing.

As shown in FIG. 2A, pramlintide formulated at pH=4 in sodium acetate buffer, similar to the commercial formulation SYMLIN®, was stable for over 100 hours under stressed conditions. Pramlintide formulated at pH=7.4 in PBS, however, aggregated after only 15±4 hrs (n=3), measured as a ˜5% change in transmittance at 540 nm, indicating a dramatic reduction in stability at physiologic pH. In contrast, when formulated with CB[7]-PEG at pH=7.4 in PBS, pramlintide remained stable well beyond pramlintide formulated at pH=7.4, with less than 2% change in transmittance at 540 nm from 0 to at least 100 hours under stressed conditions. Thus, pramlintide formulated with CB[7]-PEG pH=7.4 inhibited protein aggregation.

In addition to stabilizing pramlintide, aspart and lispro separately, CB[7]-PEG also facilitated the development of a stable insulin-pramlintide co-formulation (FIGS. 2B, 2C). Pramlintide and aspart co-formulated at pH=7.4 in PBS in the absence of CB[7]-PEG aggregated after only 2.9±0.2 hrs (aspart+pramlintide) or 4.9±0.3 hrs (lispro+pramlintide) under stressed conditions, while co-formulation with CB[7]-PEG in the same buffer conditions was completely stable for the duration of the 100-hour kinetic test. The results shown in FIG. 2B demonstrate that simultaneous supramolecular PEGylation of aspart and pramlintide with CB[7]-PEG enables the development of a viable dual hormone co-formulation. Similarly, FIG. 2C shows in vitro stability of pramlintide-lispro (1:6 and 1:20 molar ratio) co-formulations with CB[7]-PEG at physiological pH.

### Example 3: Pharmacodynamics and Pharmacokinetics of Formulations in Diabetic Rats

Having established the stability of our formulations, the efficacy of the co-formulation was evaluated in vivo by measuring blood glucose depletion in a well-studied rat model of insulin-deficient diabetes, prepared using streptozotocin (STZ) to induce pancreatic β-cell death. The object of this testing was to demonstrate that increasing the overlap between insulin and pramlintide pharmacokinetics will enable the development of a more physiologically-relevant dual-hormone treatment.

Male Sprague Dawley rats (Charles River) were used for experiments. Animal studies were performed in accordance with the guidelines for the care and use of laboratory animals; all protocols were approved by the Stanford Institutional Animal Care and Use Committee. The protocol used for STZ induction adapted from the protocol by Kenneth K. Wu and Youming Huan. Briefly, male Sprague Dawley rats 160-230 g (8-10 weeks) were weighed and fasted 6-8 hours prior to treatment with STZ. STZ was diluted to 10 mg/mL in the sodium citrate buffer immediately before injection. STZ solution was injected intraperitoneally at 65 mg/kg into each rat. Rats were provided with water containing 10% sucrose for 24 hours after injection with STZ. Rat blood glucose levels were tested for hyperglycemia daily after the STZ treatment via a tail vein blood collection using a handheld Bayer Contour Next glucose monitor (Bayer). Diabetes was defined as having 3 consecutive blood glucose measurements >400 mg/dL in non-fasted rats.

Diabetic rats were fasted for 6-8 hours. Rats were injected subcutaneously with the following formulations: (i) NOVOLOG® (1.5 U/kg), (ii) separate injections of NOVOLOG® (1.5 U/kg) and pramlintide (1:15 pramlintide to aspart; 2.3 μg/kg), (iii) insulin-pramlintide co-formulation (zinc-free aspart at 1.5 U/kg; pramlintide at 2.3 μg/kg) with CB[7]-PEG (5:1 molar ratio). Before injection, baseline blood glucose was measured. Rats with a baseline blood glucose between 400 mg/dL-500 mg/dL were selected for the study. After injection, blood glucose measurements were taken every 3 minutes for the first 30 minutes, then every 5 minutes for the next 30 minutes, then at 75, 90, 120, 150, and 180 minutes using a hand-held glucose monitor (Bayer).

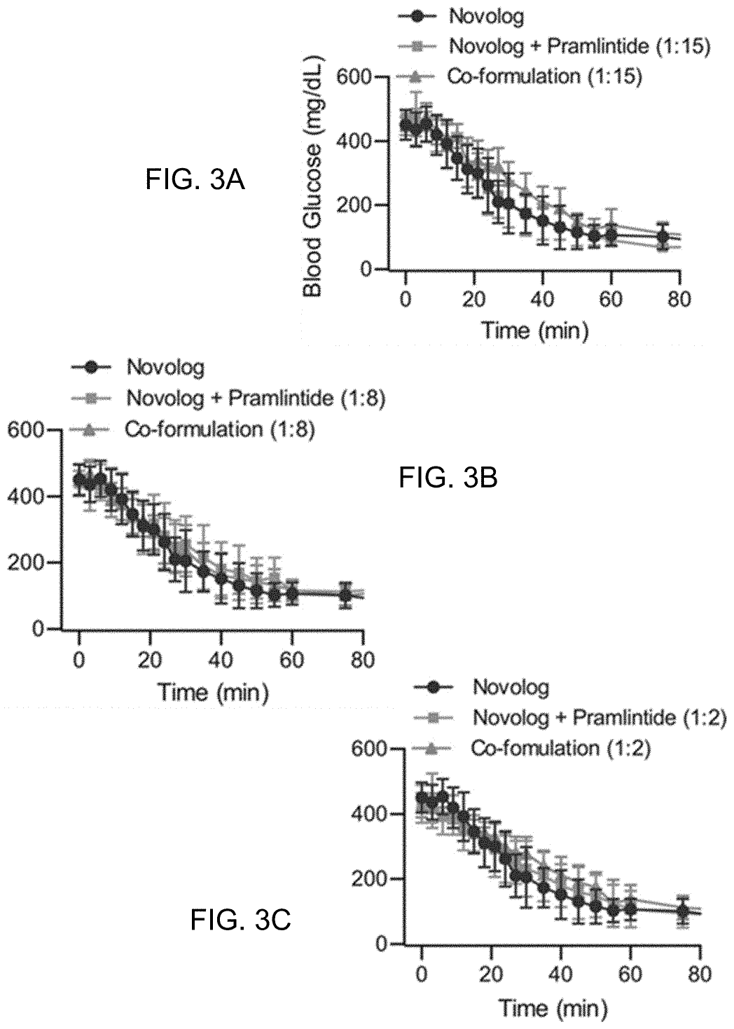

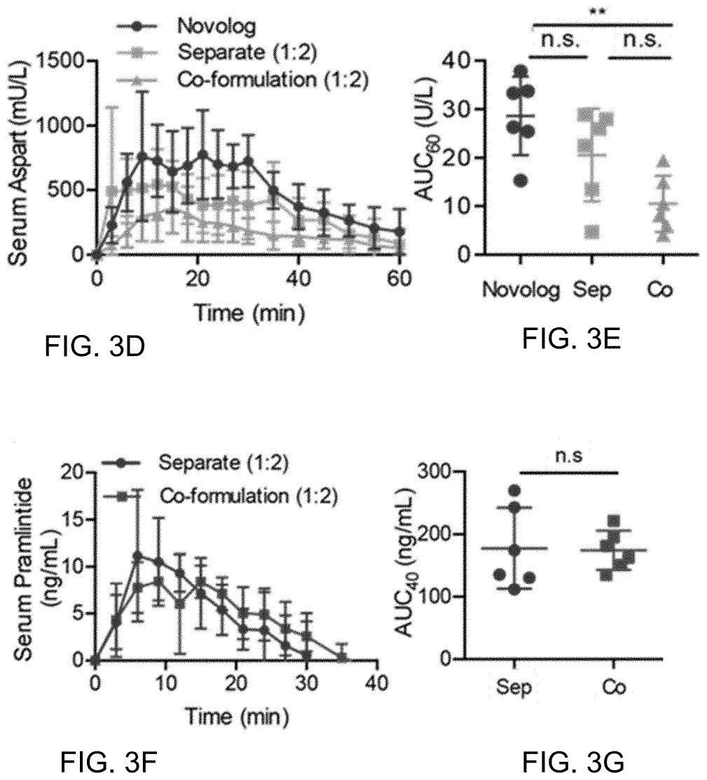

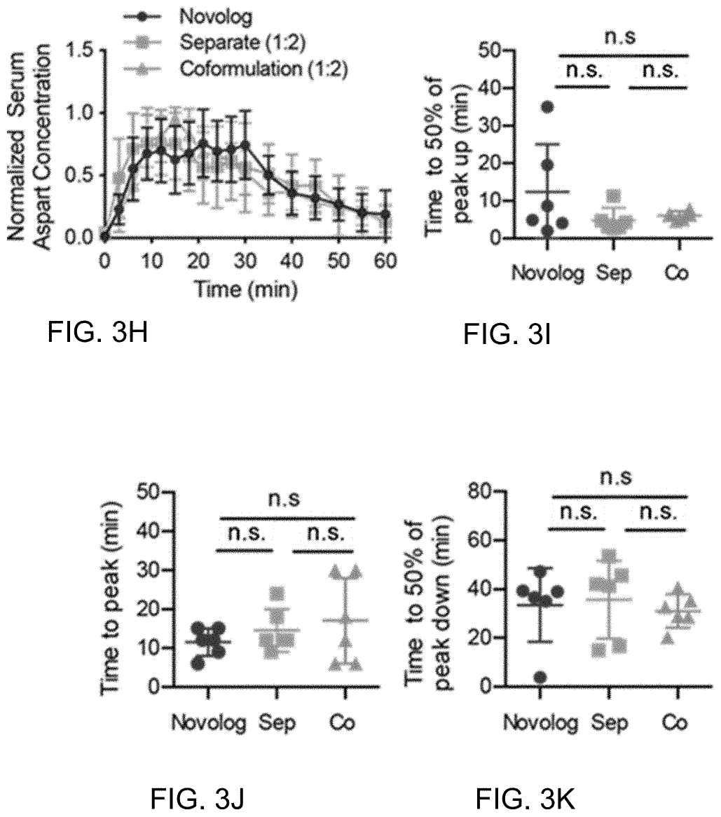

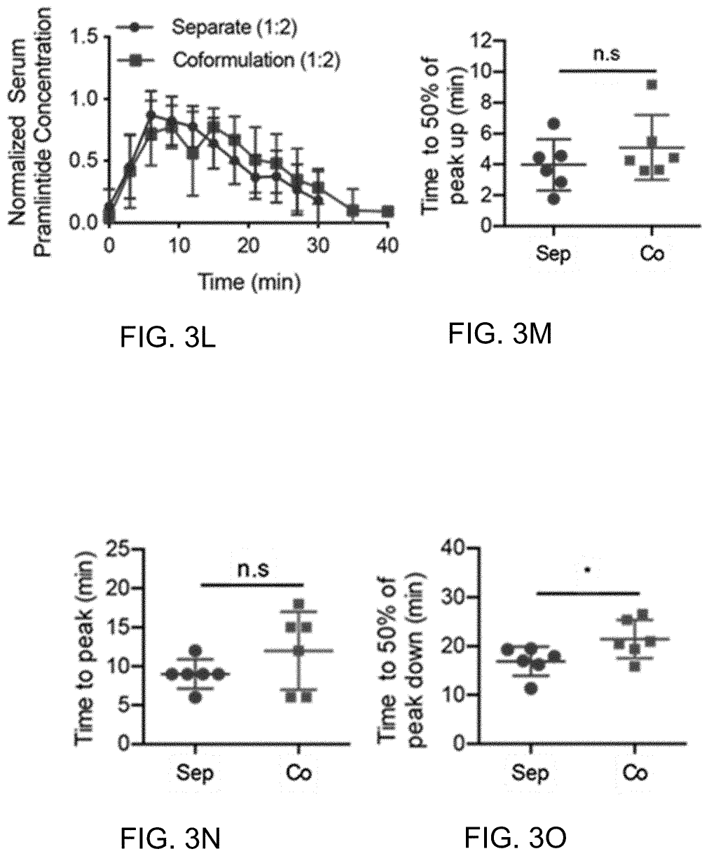

In these studies, aspart-pramlintide co-formulations (PBS at pH=7) comprising zinc-free aspart (1.5 U/kg), CB[7]-PEG (5 equivalents relative to insulin), and pramlintide (either 1:15, 1:8 or 1:2 equivalents relative to insulin) were compared to commercial NOVOLOG® alone (1.5 U/kg) and to the clinically relevant combination of NOVOLOG® (1.5 U/kg) and pramlintide (sodium acetate buffer at pH=4) administered in separate injections (FIGS. 3A-3C). The fixed molar ratios of endogenous amylin to insulin reported in the literature range from 1:20 and 1:7. Each treatment group was therefore evaluated at different molar ratios of pramlintide to insulin of 1:15, 1:8, or 1:2.

While pramlintide:insulin molar ratios of 1:15 (FIG. 3A) and 1:8 (FIG. 3B) are representative of endogenous amylin:insulin secretion, a 1:2 formulation (FIG. 3C) was also evaluated to increase the signal-to-noise ratio for in vivo pharmacokinetic studies. The rate of blood glucose depletion following administration in fasted diabetic rats was similar between aspart-pramlintide co-formulation and control groups of NOVOLOG® only or separate NOVOLOG® and pramlintide injections (n=6). The molar ratio of pramlintide to NOVOLOG® had no effect on the rate or degree of blood glucose depletion. Rats had an average baseline blood glucose of 448±17 mg/dL across all groups and blood glucose was depleted to 116±17 mg/dL by one hour after formulation injection. As the primary actions of pramlintide include slowing gastric emptying and increasing satiety as methods to slow the introduction of glucose into the blood, there was a negligible effect in blood glucose depletion between formulations when using fasted rats according to the standard protocols used here.

### Example 4: Pharmacokinetics of Insulin and Pramlintide in Diabetic Rats

Serum pramlintide concentrations were quantified using a human amylin ELISA kit (Phoenix Pharmaceuticals) with pure pramlintide as standards. Serum NOVOLOG® concentrations were quantified using a Human Insulin & Insulin Analogs ELISA kit (Alpha Diagnostics International) with NOVOLOG® standards.

Serum concentrations of insulin and pramlintide were measured over time by ELISA following subcutaneous administration of each of the treatment groups outlined above to assess the degree of overlap between the pharmacokinetic profiles of the two hormones (FIG. 3D). Aspart was administered at 1.5 U/kg in all treatment groups and the area under the curve (AUC) of serum aspart vs. time in each treatment group was calculated. Aspart AUC following the administration in co-formulation with pramlintide (10±6 mU/mL) was significantly lower than when administered alone in commercial NOVOLOG® (29±8 mU/mL; n=6) or in a separate injection to pramlintide (21±10 mU/mL; n=6) (FIG. 3E). These results suggest that pramlintide can affect aspart serum concentrations and this effect is amplified when the dual-hormone therapy is administered in a co-formulation treatment rather than in two separate injections. The “onset” rate of fast-acting insulins is often determined using two metrics: (i) time-to-50% normalized peak height up, and (ii) time-to-peak insulin serum concentration. Normalized serum concentration data was used to compare the time-to-peak aspart concentrations between treatment groups (FIG. 3H). Peak aspart concentrations occur 19±10 min following subcutaneous administration of commercial NOVOLOG® alone, 10±5 min following NOVOLOG® alongside a separate injection to pramlintide and 15±5 min after administration in a single co-formulation injection. No difference was seen in aspart time to peak (FIG. 3J), or time-to-50% normalized peak height up following administration of commercial NOVOLOG® alone, NOVOLOG® alongside a separate injection of pramlintide, or administration in a single co-formulation injection (FIG. 3I-3J). Similarly, there was no significant difference in aspart duration-of-action, determined by measuring the terminal time-to-50% normalized peak height, between treatment groups (FIG. 3K).

When evaluating pramlintide pharmacokinetics, no significant differences were seen in pramlintide AUC following different treatments (FIGS. 3F, 3G). Peak serum pramlintide concentration was 8±2 min when pramlintide and insulin are administered in separate injections compared to 10±6 minutes in the insulin-pramlintide co-formulation (FIGS. 3M, 3N), indicating that there was no significant difference in time to pramlintide onset. In contrast, pramlintide duration-of-action was extended from 17±3 min for separate administrations to 21±4 min for the co-formulation (FIG. 3O). Error bars indicate mean±s.d. with n=6 for all groups. Statistical significance was determined by a two-tailed student's t-test.

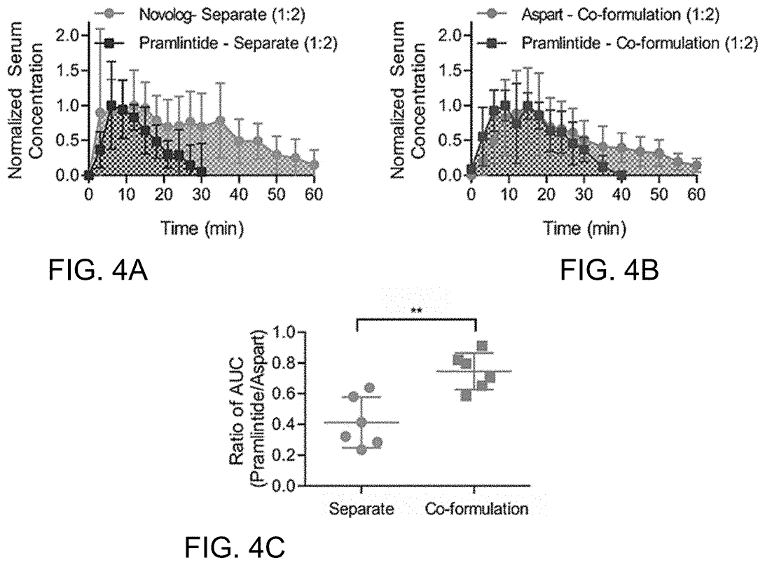

The combination of shorter insulin duration of action and longer pramlintide duration of action observed in the aspart-pramlintide co-formulation is likely a result of the more similar molecular size of the aspart/CB[7]-PEG and pramlintide/CB[7]-PEG complexes. This feature may enable greater pharmacokinetic matching of the two therapeutics when compared to administration in separate injections. The overlap of the pharmacokinetic profiles can be represented by the ratio of AUC of serum pramlintide to serum aspart, which was determined to be 0.7±0.1 when these proteins are delivered in co-formulation, but only 0.4±0.2 (n=6) when delivered in separate injections. FIGS. 4A and 4B show the mean normalized serum concentration (normalized for each individual rat) of NOVOLOG® and pramlintide when administered as two separate injections (FIG. 4A) or pramlintide-aspart co-formulation with CB[7]-PEG at physiologic pH (FIG. 4B). FIG. 4C plots the ratio of the area under the curve (AUC) of the pharmacokinetic profiles of pramlintide and aspart for administration as separate injections and as a co-formulation (**p=0.0025). Error bars indicate mean±s.d. with n=6 for all groups. Statistical significance was determined by a two-tailed student's t-test.

### Example 5: Pharmacodynamics and Pharmacokinetics of Formulations in Diabetic Pigs

Previous studies in rodent models of insulin-deficient diabetes have observed a consistent time-to-peak onset between rapid-acting insulin analogues and regular insulin. This contrasts with human studies in which “rapid-acting” insulin formulations exhibit time-to-onset that is reduced roughly by half. The difference between the pharmacokinetics observed in rats and humans (or pigs) arises on account of two important differences in the animal models: (i) the dilution of the formulations requires to enable accurate dosing in rats, and (ii) differences in absorption from the subcutaneous space arising from physiological differences between these species. First, the size of the rat necessitates dilution of insulin formulations to facilitate administration of an accurate dose. Dilution of insulin shifts the equilibrium of the insulin association states and favors the monomeric and dimeric forms of insulin as opposed to the hexameric form. In contrast, pramlintide only exists in monomeric form and is unaffected by dilution. Secondly, rats have loose skin that facilitates more rapid absorption of administered compounds following s.c. administration due to the greater surface area for absorption.

In contrast, pigs are large enough for insulin to be administered accurately using standard concentrations (100 U/mL), ensuring the observed pharmacokinetics are not skewed by dilution effect. Pigs also have tight skin and subcutaneous tissue that is similar to humans, making them the most relevant preclinical model for studying pharmacokinetics of biopharmaceuticals following s.c. administration. While the pharmacokinetics of pramlintide in pigs is similar to that in humans, insulin exhibits shorter duration of action in pigs—2 hrs vs. 4 hrs in humans.

Female Yorkshire pigs (Pork Power) were used for experiments. Animal studies were performed in accordance with the guidelines for the care and use of laboratory animals and all protocols were approved by the Stanford Institutional Animal Care and Use Committee. Type-1-like diabetes was induced in pigs (25-30 kg) using streptozotocin (STZ) (MedChemExpress). STZ was infused intravenously at a dose of 125 mg/kg and animals were monitored for 24 hours. Food and administration of 5% dextrose solution was given as needed to prevent hypoglycemia. Diabetes was defined as fasting blood glucose greater than 300 mg/dL.

To assess translationally-relevant pharmacokinetics and post-prandial treatment benefits (See Supplemental Information) of insulin-pramlintide co-formulations, we conducted studies in a swine model of insulin-deficient diabetes. Initially, fasted diabetic pigs were treated with either (i) commercial HUMALOG® (4 U; 0.13 U/kg), (ii) separate injections of commercial HUMALOG® (4 U; 0.13 U/kg) and pramlintide (pH=4; pramlintide:insulin ratio of 1:6), or (iii) lispro-pramlintide co-formulation with CB[7]-PEG (4 U insulin; pramlintide:insulin ratio of 1:6). Treatments were administered simultaneously with a 200 g meal.

Insulin lispro was chosen for these studies due to greater availability of insulin lispro (HUMALOG®) at the time of experiments. EDTA was removed from formulations given to pigs using a desalting column. Before injection, baseline blood was sampled from an intravenous catheter line and measured using a handheld glucose monitor (Bayer Contour Next). After injection, blood was sampled from the intravenous catheter line every 5 minutes for the first 60 minutes, then every 30 minutes up to 4 hours. Blood glucose was measured using a handheld blood glucose monitor and additional blood was collected in serum tubes (Starstedt) or K₂EDTA plasma tubes (Greiner-BioOne) for analysis with ELISA. Serum and plasma lispro concentrations were quantified using an iso-insulin ELISA kit or lispro-NL ELISA kit (Mercodia), serum and plasma pramlintide was quantified using a human amylin ELISA kit (Millipore Sigma), and serum and plasma glucagon was quantified with a Glucagon ELISA kit (Mercodia). If the ELISA of a sample was run multiple times the averages of the values was taken for analysis.

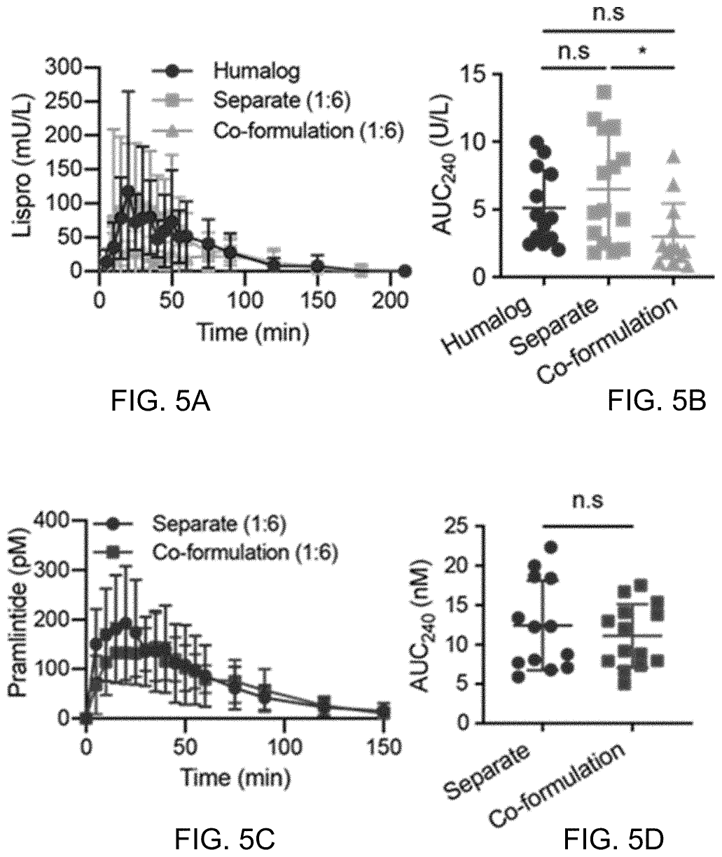

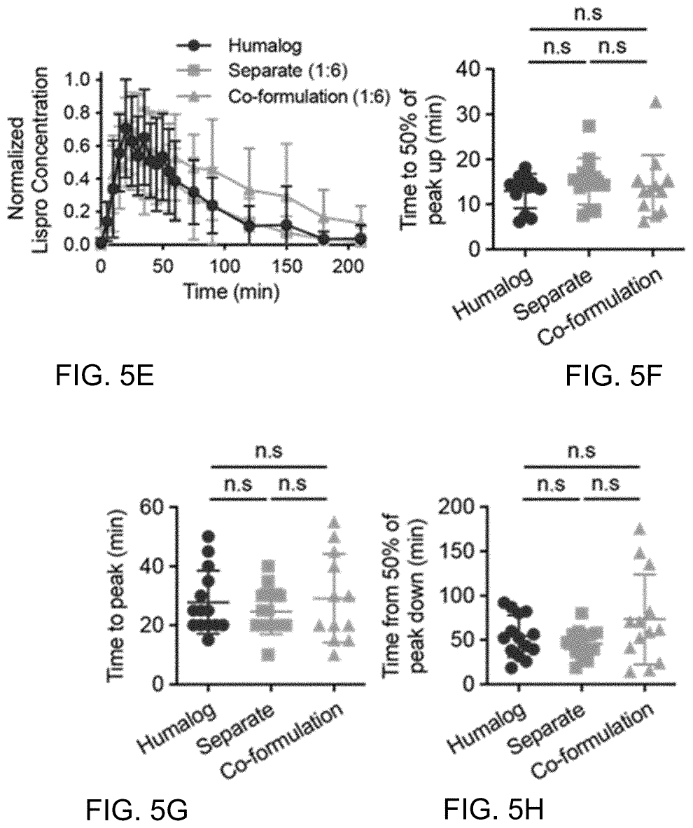

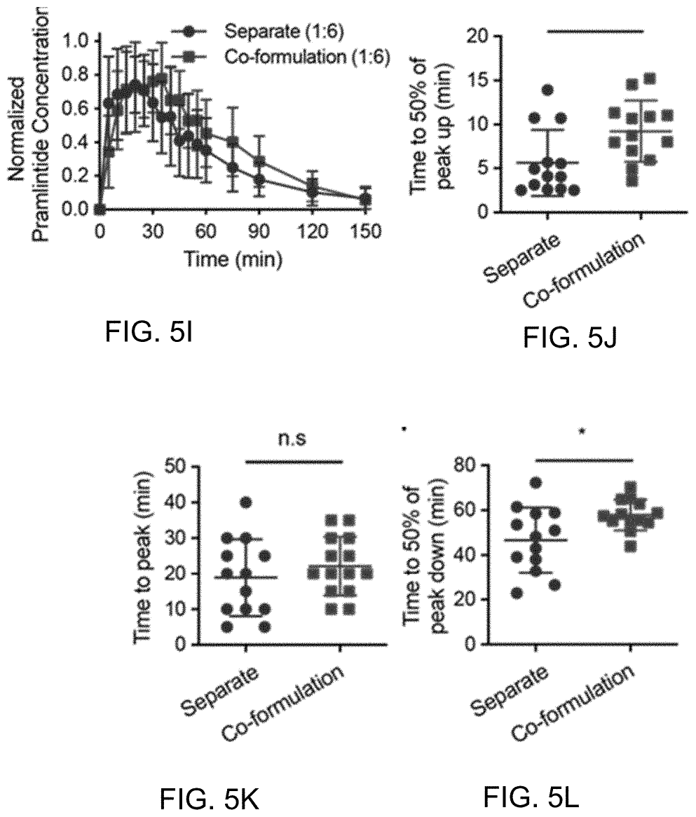

FIGS. 5A and 5C, respectively, show the pharmacokinetics of insulin lispro in mU/L, and pramlintide in pM. The area under the pharmacokinetic curves (AUC) of for lispro (*p=0.017) and pramlintide for the first 240 minutes after subcutaneous injection are shown in FIGS. 5B and 5D, respectively. Pharmacokinetics for each pig were individually normalized to peak concentrations and normalized values were averaged for lispro concentration (FIG. 5E) or pramlintide concentration (FIG. 51) for each treatment group. FIGS. 5F and 5J plot the time to reach 50% of peak lispro and pramlintide (*p=0.018) concentration (onset), respectively, while FIGS. 5G and 5K plot time to reach peak lispro or pramlintide concentration. FIGS. 5H and 5L show time for lispro and pramlintide (*p=0.019) depletion to 50% of peak concentration. Error bars indicate mean±s.d., n=12-15 for all groups. The Grubbs' outlier test (alpha=0.05) was used to remove outliers. Statistical significance was determined by a two-tailed student's t-test.

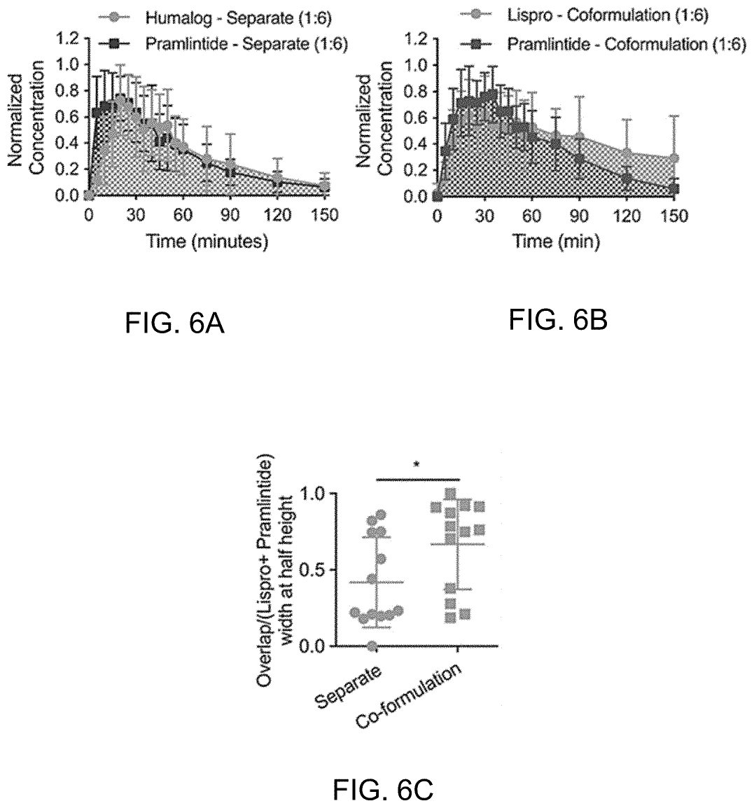

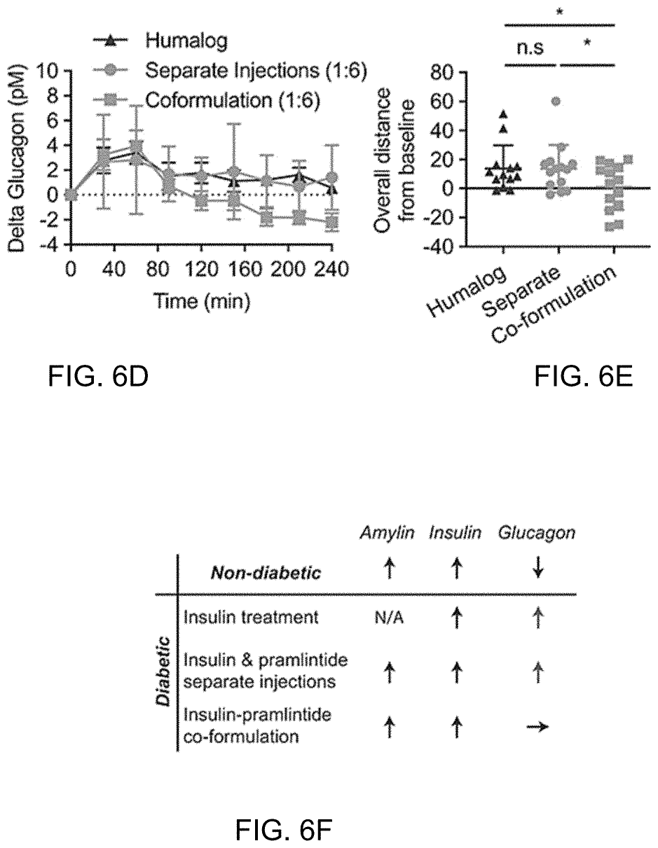

The modulation of the pramlintide pharmacokinetics was confirmed by an increase in overlap between insulin and pramlintide pharmacokinetic curves when administered as a co-formulation compared to administration in separate injections as shown in FIGS. 6A-6B, which plot the mean normalized concentration (normalized individually for each pig) of lispro and pramlintide when administered as two separate injections (FIG. 6A) or as a co-formulation with CB[7]-PEG (FIG. 6B). The overlap between curves was evaluated as the time during which both lispro and pramlintide concentrations were greater than 0.5 (width at half peak height), shown in FIG. 6C as a ratio of overlap time over the total width of both peaks (Overlap/(Lispro+Pramlintide−Overlap).) The ratio of the overlap time over the total time at half-peak height was determined to be 0.67±0.29 for the co-formulation and 0.42±0.30 for separate injections.

According to the initial hypothesis results, the increased overlap of insulin and pramlintide pharmacokinetics in the insulin-pramlintide co-formulation should be advantageous for treatment outcomes. As demonstrated by this testing, co-formulation resulted in suppressed post-prandial glucagon levels (1±16 pM) compared to both HUMALOG® alone (14±16 pM) as well as insulin and pramlintide delivered in two separate injections (14±17 pM), as shown in FIGS. 6D and 6E. Error bars indicate mean s.d. with n=13-14 for all groups. The ROUT test (Q=1%) was used to remove outliers. Statistical significance was determined by a two-tailed student's t-test.

Separate injections did not result in statistically significant differences in post-prandial glucagon suppression compared to HUMALOG® alone. These results suggest that co-formulation improves restoration of metabolic signaling compared to separate delivery of insulin and pramlintide. FIG. 6F provides a summary schematic of how treatment variations affects post-prandial glucagon.

### Example 6: CB [7]-PEG Biocompatibility in Rats

As CB[7]-PEG is a new chemical entity, biocompatibility was assessed by using blood chemistry and histopathology to look for negative effects on the liver or kidney. Healthy Sprague Dawley rats (n=4) received daily injections of CB[7]-PEG (at a dose equivalent to what would be administered in an insulin injection—0.2 mg/kg in PBS (pH=7.4) for six weeks. Blood chemistry was monitored biweekly (days 14, 28 and 42) and single-blinded assessment of the histopathology of the liver and kidney was conducted at the end-point of the study. A control group of healthy rats housed under the same conditions, but who received no injections, were used to control for the impact of aging on blood chemistry values.

Chemistry analysis is performed on the Siemens Dimension Xpand analyzer. A medical technologist performed all testing, including dilutions and repeat tests as indicated, and reviewed all data. At the end of the six week experiment the rats were euthanized and kidneys and liver were collected for histology. Harvested tissue was fixed and then transverse sections of the left lateral lobe and right medial lobe of the liver were taken and longitudinal sections of the kidney for histology (n=2). Hemotoxylin & Eosin and Masson's Trichrome staining were performed by Histo-tec Laboratory.

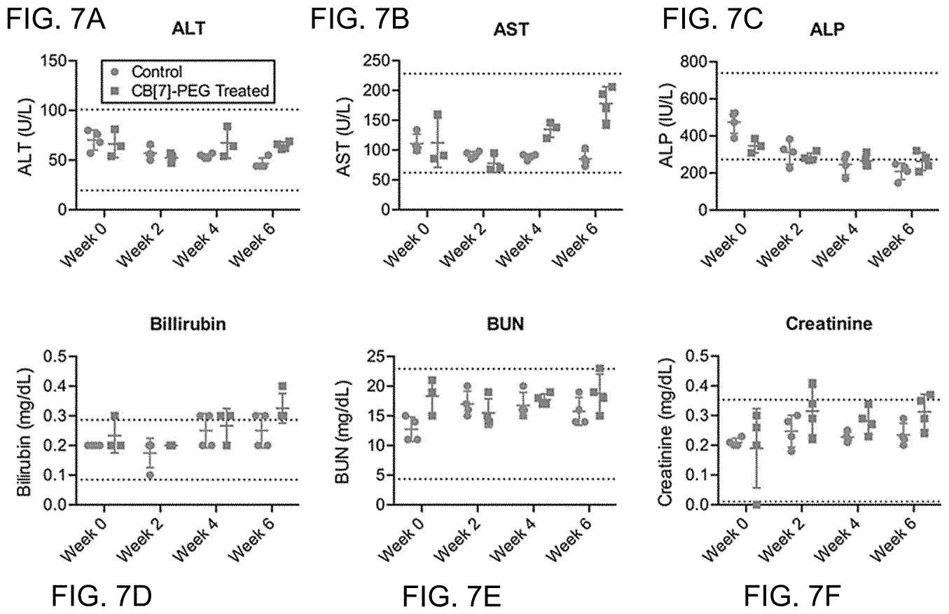

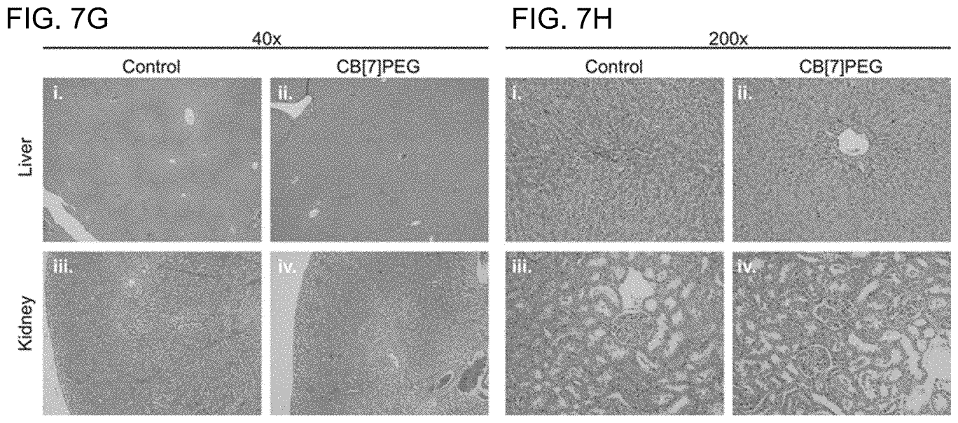

Blood chemistry for both treatment and control groups were compared to a healthy population of Sprague Dawley rats (age 8-9 weeks). Liver toxicity was assessed through measurement of alanine aminotransferase (ALT), aspartate aminotransferase (AST), alkaline phosphotase (ALP), and bilirubin. Kidney toxicity was evaluated by examining creatinine and blood urea nitrogen (BUN) levels. As shown in FIGS. 7A-7F, values for ALT, AST, ALP, creatine, and BUN were within the range of healthy rats (defined as the mean±2 standard deviations) for both the treatment and control groups.

A single-blind assessment of liver and kidney histology sections from treated and control rats was performed by a pathologist at the endpoint of the study. Haemotoxylin and Eosin (H&E) and Masson's Trichrome staining were performed and no differences in fat content or fibrosis between the treated and untreated rats was observed in either tissue (FIGS. 7G-7H). Liver portal triads in both control and treated samples appeared normal with no fibrosis. Some focal fat was observed in both treated and control tissues. No fibrosis was observed in either treated or healthy kidney samples.

### Example 7: CB [7]-PEG Biocompatibility in Pigs

Evaluation of blood chemistry in diabetic pigs after repeated treatment with CB[7]-PEG during the course of the study corroborated the observations made in rats. Pigs were dosed with insulin-pramlintide co-formulation (1:6) containing CB[7]-PEG at 10-13 meals over the course of six weeks. Week 0 measurements were taken 3-4 days following the induction of diabetes, and Week 6 measurements were taken at the end of the study.

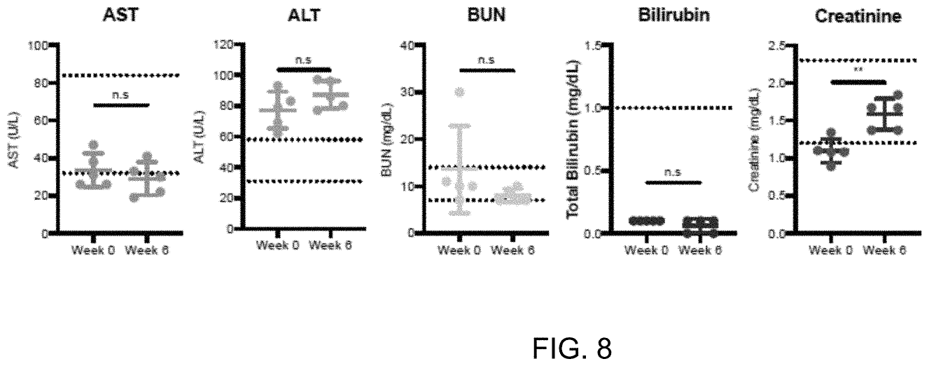

Measured values for AST, ALT, BUN, bilirubin and creatinine are shown in FIG. 8. The dotted lines in each plot indicate that the range of values observed in treated animals is within the normal range for healthy pigs. It should be noted that diabetes induction with streptozoticin can cause liver and kidney damage that causes blood chemistry values to deviate outside the normal range. A student's t-test was used to evaluate statistical significance between time points. The only marker that demonstrates statistically significant difference from the beginning to the end of the study is creatinine (p=0.001), however, the Week 6 measurements fall well within the normal range.

### Additional Observations

Natural insulin secretion results in insulin levels that are several times higher in the liver than in the peripheral tissues due to first pass insulin absorption from the portal vein. While subcutaneous insulin replacement therapy successfully stimulates glucose uptake in the peripheral tissues, it does not suppress hepatic glucose secretion to the same degree as endogenous insulin as a result of differential pharmacokinetics, pharmacodynamics, and biodistribution. In turn, the reduction in hepatic signaling results in unrestricted glycogen mobilization in the post-prandial period. A physiological replacement therapy for amylin in diabetic patients may play an important role in improving the efficacy of insulin treatments since amylin and its analogues act synergistically to inhibit glycogen mobilization from hepatic tissues by suppressing post-prandial glucagon. Notwithstanding a clear need, co-formulation of biopharmaceuticals is difficult due to their poor stability and potential for differential solubility—traditional formulation approaches to prepare an insulin-pramlintide co-formulation have been unsuccessful.

As disclosed herein, a co-formulation of insulin and pramlintide was created using an approach that utilizes simultaneous supramolecular PEGylation of the two hormones with CB[7]-PEG to stabilize pramlintide in combination with insulin analogues such as aspart or lispro in the absence of formulation zinc. The utility of this excipient-based approach was demonstrated to simultaneously endow otherwise incompatible proteins, insulin and pramlintide, with PEG chains to inhibit protein aggregation. This approach exploits the specific and strong binding of the macrocyclic host molecule CB[7] to aromatic amino acids, including the N-terminal phenylalanine on insulin and the amidated C-terminal tyrosine on pramlintide, through simple mixing as these interactions are non-covalent. CB[7]-PEG exhibits binding affinities for these proteins in the micromolar range such that over 98% of the complexes are bound at typical formulation concentrations, while less than 1% are bound upon dilution following administration in the body. This feature affords the automatic release of authentic, unmodified therapeutic proteins upon administration and overcomes the limitations of traditional approaches to covalent grafting of polymers onto proteins, which include reduced activity. The inventive approach thereby offers a broadly useful and modular excipient strategy for formulation of unmodified protein drugs to enhance their formulation shelf life and alter pharmacokinetics.

The simultaneous supramolecular PEGylation of insulin and pramlintide not only enabled their co-formulation at physiologic pH by enhancing the stability of the two proteins, but also facilitates the modification of insulin-pramlinitde pharmacokinetics to more closely mimic endogenous hormone secretion and restore meal-time glucagon suppression. When injected separately according to the current clinical model, fast-acting insulin analogues and pramlintide have reduced overlap between their pharmacokinetic curves resulting from the slower absorption of insulin as traditionally formulated (i.e., consisting of a combination of monomers, dimers, and hexamers) from the subcutaneous space than the pramlintide, which only exists in a monomeric form. Using a translationally-relevant porcine model of insulin-deficient diabetes, meal-time administration of an insulin-pramlintide co-formulation was demonstrated to lead to increased overlap of insulin and pramlintide pharmacokinetics and restoration of mealtime glucagon suppression when compared with the clinical standard of separate administration of the hormones. While separate delivery of pramlintide has been clinically shown to suppress meal-time glucagon at high doses, insulin-amylin co-formulation is shown to exhibits potent glucagon suppression at lower doses than can be achieved with separate administrations. Co-formulation, therefore, captures the synergistic effects of amylin and insulin and shows promise as a true biomimetic dual-hormone replacement therapy with greater physiological relevance than insulin alone. Moreover, the ability of this biomimetic dual-hormone treatment therapy to be administered in a single injection will reduce patient burden and potentially enable more broad adoption by patients who would benefit from such a therapy in the treatment of both Type 1 and Type 2 diabetes.

It is to be understood that the compositions, processes and embodiments described herein are not intended to limit the scope of the invention to particular formulations or process parameters. It is also to be understood that the terminology used herein is for the purpose of describing particular embodiments of the invention only, and is not intended to be limiting.

### REFERENCES (INCORPORATED HEREIN BY REFERENCE)

1. WHO. Diabetes: Key Facts. World Health Organization. (2017).
2. Borm, A. K. et al. The effect of pramlintide (amylin analogue) treatment on bone metabolism and bone density in patients with type 1 diabetes mellitus. Horm. Metab. Res. 31, 472-475 (1999).
3. Gottlieb, A. et al. Pramlintide as an adjunct to insulin therapy improved glycemic and weight control in people with type 1 diabetes during treatment for 52 weeks. Diabetes. 49, A109-A109 (2000).
4. Ryan, G. J., Jobe, L. J. & Martin, R. Pramlintide in the treatment of type 1 and type 2 diabetes mellitus. Clin. Ther. 27, 1500-1512 (2005).
5. Edelman, S. et al. A double-blind, placebo-controlled trial assessing pramlintide treatment in the setting of intensive insulin therapy in type 1 diabetes. Diabetes Care. 29, 2189-2195 (2006).
6. Jones, M. C. Therapies for diabetes: pramlintide and exenatide. Am Fam Physician. 75, 1831-1835 (2007).
7. Rodriguez, L. M. et al. The role of prandial pramlintide in the treatment of adolescents with type 1 diabetes. Pediatr. Res. 62, 746-749 (2007).
8. Weinzimer, S. A. et al. Effect of pramlintide on prandial glycemic excursions during closed-loop control in adolescents and young adults with type 1 diabetes. Diabetes Care. 35, 1994-1999 (2012).
9. Grunberger, G. Novel therapies for the management of type 2 diabetes mellitus: part 1. pramlintide and bromocriptine-QR. J. Diabetes. 5, 110-117 (2013).
10. Hay, D. L. et al. Amylin: pharmacology, physiology, and clinical potential. Pharmacol. Rev. 67, 564 (2015).
11. Wang, H. et al. Rationally designed, nontoxic, nonamyloidogenic analogues of human islet amyloid polypeptide with improved solubility. Biochemistry-US. 53, 5876-5884 (2014).
12. Whitehouse, F. et al. A randomized study and open-label extension evaluating the long-term efficacy of pramlintide as an adjunct to insulin therapy in type 1 diabetes. Diabetes Care. 25, 724-730 (2002).
13. Ratner, R. E. et al. Amylin replacement with pramlintide as an adjunct to insulin therapy improves long-term glycaemic and weight control in type 1 diabetes mellitus: a 1-year, randomized controlled trial. Diabetic Med. 21, 1204-1212 (2004).
14. Hampp, C. et al. Use of antidiabetic drugs in the U.S., 2003-2012. Diabetes Care. 37, 1367-1374 (2014).
15. Martin, C. The physiology of amylin and insulin: maintaining the balance between glucose secretion and glucose uptake. Diabetes Educator. 32, 101S-104S (2006).
16. Heptulla, R. A. et al. The role of subcutaneous pramlintide infusion in the treatment of adolescents with type 1 diabetes. Diabetes. 54, A110-A111 (2005).
17. Want, L. L. & Ratner, R. Exenatide and pramlintide: new therapies for diabetes. Int. J. Clin. Pract. 60, 1522-1523 (2006).
18. Gast, K. et al. Rapid-acting and human insulins: hexamer dissociation kinetics upon dilution of the pharmaceutical formulation. Pharm. Res. 34, 2270-2286 (2017).
19. Holleman, F.& Hoekstra, J. B. L. Insulin Lispro. New Engl. J. Med. 337, 176-183 (1997).
20. Riddle, M. C. et al. Fixed ratio dosing of pramlintide with regular insulin before a standard meal in patients with type 1 diabetes. Diabetes Obes. Metab. 17, 904-907 (2015).
21. Haidar, A. et al. Insulin-plus-pramlintide artificial pancreas in type 1 diabetes—randomized controlled trial. Diabetes. 67, (2018).
22. Riddle, M. C. et al. Control of postprandial hyperglycemia in type 1 diabetes by 24-hour fixed-dose coadministration of pramlintide and regular human insulin: a randomized, two-way crossover study. Preprint at https://doi.org/10.2337/dc18-1091 (2018).
23. Manning, M. C. et al. Stability of protein pharmaceuticals: an update. Pharm. Res. 27, 544-575 (2010).
24. Mitragotri, S., Burke, P. A. & Langer, R. Overcoming the challenges in administering biopharmaceuticals: formulation and delivery strategies. Nat. Rev. Drug Discov. 13, 655-672 (2014).
25. Yang, C., Lu, D. & Liu, Z. How PEGylation enhances the stability and potency of insulin: amolecular dynamics simulation. Biochemistry-US. 50, 2585-2593 (2011).
26. Guerreiro, L. H. et al. Preparation and characterization of PEGylated amylin. AAPS PharmSciTech. 14, 1083-1097 (2013).
27. Sisnande, T. et al. Monoconjugation of human amylin with methylpolyethyleneglycol. PLoS One. 10, e0138803 (2015).
28. Veronese, F. M. & Mero, A. The impact of PEGylation on biological therapies. BioDrugs. 22, 315-29 (2008).
29. Webber, M. J. et al. Supramolecular PEGylation of biopharmaceuticals. P. Natl. Acad. Sci. USA. 113, 14189-14194 (2016).
30. Hirotsu, T. et al. Self-assembly PEGylation retaining activity (SPRA) technology via a host-guest interaction surpassing conventional PEGylation methods of proteins. Mol. Pharm. 14, 368-376 (2017).
31. Bush, M., Bouley, N. & Urbach, A. R. Charge-mediated recognition of N-terminal tryptophan in aqueous solution by a synthetic host. J. Am. Chem. Soc. 127, 14511-14517 (2005).
32. Heitmann, L. M. et al. Sequence-specific recognition and cooperative dimerization of N-terminal aromatic peptides in aqueous solution by a synthetic host. J. Am. Chem. Soc. 128, 12574-12581 (2006).
33. Rajgariah, P.& Urbach, A. R. Scope of amino acid recognition by cucurbit[8]uril. J. Incl. Phenom. Macro. 62, 251-254 (2008).
34. Reczek, J. J. et al. Multivalent recognition of peptides by modular self-assembled receptors. J. Am. Chem. Soc. 131, 2408-2415 (2009).
35. Berthon, G. Handbook Of Metal-ligand Interactions in Biological Fluids: Bioinorganic chemistry. (New York: Marcel Dekker, 1995).
36. Waters, R. S. et al. EDTA chelation effects on urinary losses of cadmium, calcium, chromium, cobalt, copper, lead, magnesium, and zinc. Biol. Trace. Elem. Res. 83, 207-221 (2001).
37. Hvidt, S. Insulin association in neutral solutions studied by light scattering. Biophys. Chem. 39, 205-213 (1991).
38. Fineberg, S. E. et al. Immunological responses to exogenous insulin. Endocr. Rev. 28, 625-652 (2007).
39. Woods, R. J. et al. Intrinsic fibrillation of fast-acting insulin analogs. J. Diabetes Sci. Technol. 6, 265-276 (2012).
40. da Silva, D. C. et al. Amyloidogenesis of the amylin analogue pramlintide. Biophys. Chem. 219, 1-8 (2016).
41. Like, A. A. & Rossini, A. A. Streptozotocin-induced pancreatic insulitis: new model of diabetes mellitus. Science. 193, 415-417 (1976).
42. Sanke, T. et al. Plasma islet amyloid polypeptide (Amylin) levels and their responses to oral glucose in type 2 (non-insulin-dependent) diabetic patients. Diabetologia. 34, 129-132 (1991).
43. Gedulin, B. R., Rink, T. J. & Young, A. A. Dose-response for glucagonostatic effect of amylin in rats. Metabolis. 46, 67-70 (1997).
44. Knadler, M. P. et al. Addition of 20-kDa PEG to insulin lispro alters absorption and decreases clearance in animals. Pharm. Res. 33, 2920-2929 (2016).
45. Yin, H. & Wang, R. Applications of cucurbit[n]urils (n=7 or 8) in pharmaceutical sciences and complexation of biomolecules. Isr. J. Chem. 58, 188-198 (2018).
46. Fruijtier-Pölloth, C. Safety assessment on polyethylene glycols (PEGs) and their derivatives as used in cosmetic products. Toxicology. 214, 1-38 (2005).
47. Webster, R. et al. PEGylated proteins: evaluation of their safety in the absence of definitive metabolism studies. Drug Metab. Dispos. 35, 9-16 (2007).
48. Hettiarachchi, G. et al. Toxicology and drug delivery by cucurbit[n]uril type molecular containers. PLoS One. 5, e10514 (2010).
49. Kuok, K. I. et al. Cucurbit[7]uril: an emerging candidate for pharmaceutical excipients. Ann. NY. Acad. Sci. 1398, 108-119 (2017).
50. Uzunova, V. D. et al. Toxicity of cucurbit[n]uril and cucurbit[8]uril: an exploratory in vitro and in vivo study. Org. Biomol. Chem. 8, 2037-2042 (2010).
51. FAO/WHO. Evaluation of certain food additives. Twenty-third report of the joint FAO/WHO expert committee on food additives. World Health Organ Tech. Rep. Ser. No. 648, (1980).
52. Plum, A., Agersø, H. & Andersen, L. Pharmacokinetics of the rapid-acting insulin analog, insulin aspart, in rats, dogs, and pigs, and pharmacodynamics of insulin aspart in pigs. Drug Metab. Dispos. 28, 155-160 (2000).
53. Heinemann, L. et al. Variability of the metabolic effect of soluble insulin and the rapid-acting insulin analog insulin aspart. Diabetes Care. 21, 1910-1914 (1998).
54. Hinshaw, L. et al. Effects of delayed gastric emptying on postprandial glucose kinetics, insulin sensitivity, and β-cell function. Am. J. Physiol-Endoc. M 307, E494-E502 (2014).
55. Astrazeneca Pharmaceuticals, L. P. SYMLIN (pramlintide acetate) injection. FDA.
(2014).
56. Ahrén, B. & Thorsson, O. Increased insulin sensitivity is associated with reduced insulin and glucagon secretion and increased insulin clearance in man. J. Clin. Endocr. Metab. 88, 1264-1270 (2003).
57. Chinai, J. M. et al. Molecular recognition of insulin by a synthetic receptor. J. Am. Chem. Soc. 133, 8810-8813 (2011).

1. A pharmaceutical composition comprising:
amylin or an amylin analogue;
insulin or an insulin analogue; and
a cucurbit [7] uril (CB[7])-poly(ethylene glycol) (PEG) conjugate in an effective amount sufficient to inhibit formation of amyloid fibrils.

2. The pharmaceutical composition of claim 1, wherein the amylin analogue is pramlintide.

3. The pharmaceutical composition of claim 1, wherein the insulin analogue is insulin aspart.

4. The pharmaceutical composition of claim 1, wherein the insulin analogue is insulin lispro.

5. The pharmaceutical composition of claim 1, wherein the CB[7]-PEG prevents protein precipitation for at least 100 hours.

6. The pharmaceutical composition of claim 1, wherein the insulin or insulin analogue is zinc free.

7. The pharmaceutical composition of claim 1, wherein the insulin or insulin analogue is formulated with ethylenediaminetetraacetic acid (EDTA) to remove formulation zinc.

8. The pharmaceutical composition of claim 7, where the EDTA is added at a molar ratio to zinc of 1:1.

9. The pharmaceutical composition of claim 1, wherein each of the amylin or amylin analogue and the insulin or insulin analog have similar hydrodynamic sizes.

10. A pharmaceutical composition comprising:
a co-formulation formed by simultaneous supramolecular PEGylation at physiological pH of amylin or an amylin analogue and insulin or an insulin analogue with CB[7]-PEG in the absence of formulation zinc.

11. The pharmaceutical composition of claim 10, wherein the amylin analogue is pramlintide.

12. The pharmaceutical composition of claim 10, wherein the insulin analogue is insulin aspart.

13. The pharmaceutical composition of claim 10, wherein the insulin analogue is insulin lispro.

14. The pharmaceutical composition of claim 10, wherein the CB[7]-PEG prevents protein precipitation for at least 100 hours.

15. The pharmaceutical composition of claim 10, wherein the insulin or insulin analogue is zinc free.

16. The pharmaceutical composition of claim 10, wherein the insulin or insulin analogue is formulated with ethylenediaminetetraacetic acid (EDTA) to remove formulation zinc.

17. The pharmaceutical composition of claim 16, where the EDTA is added at a molar ratio to zinc of 1:1.

18. The pharmaceutical composition of claim 10, wherein each of the amylin or amylin analogue and the insulin or insulin analog have similar hydrodynamic sizes.

19. A pharmaceutical composition comprising:
amylin or an amylin analogue; and
a cucurbit [7]uril (CB[7])-poly(ethylene glycol) (PEG) conjugate in an effective amount sufficient to inhibit formation of amyloid fibrils.

20. The pharmaceutical composition of claim 19, wherein the amylin analogue is pramlintide.

21. A method of treating a subject for diabetes, the method comprising administering a therapeutically effective amount of the pharmaceutical composition of claim 1 to the subject.

22-23. (canceled)

24. The method of claim 21, wherein the subject is human.

25-26. (canceled)

27. A method of treating a subject for diabetes, the method comprising administering a therapeutically effective amount of the pharmaceutical composition of claim 10 to the subject.

28. The method of claim 27, wherein the subject is human.

29. A method of treating a subject for diabetes, the method comprising administering a therapeutically effective amount of the pharmaceutical composition of claim 19 to the subject.

30. The method of claim 29, wherein the subject is human.
